SEQUENCE LISTING (30 sequences)

SEQ ID NO: 1 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 2 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 3 - protein, 159 aa, Homo sapiens
MALETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 4 - protein, 180 aa, Homo sapiens
MALADLYEEGGGGGGEGEDNADSKETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 5 - protein, 143 aa, Homo sapiens
MQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 6 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 7 - protein, 133 aa, Homo sapiens
MFHVSFRYIFGLPPLILVLLPVASSDCDIEGKDGKQYESVLMVSIDQLLDSMKEIGSNCLNNEFNFFKRHICDANKVKGRKPAALGEAQPTKSLEENKSLKEQKKLNDLCFLKRLLQEIKTCWNKILMGTKEH

SEQ ID NO: 8 - protein, 64 aa, Homo sapiens
MKEIGSNCLNNEFNFFKRHICDANKEENKSLKEQKKLNDLCFLKRLLQEIKTCWNKILMGTKEH

SEQ ID NO: 9 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 10 - protein, 102 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKAEVPENRGMDS

SEQ ID NO: 11 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 12 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 13 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 14 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 15 - protein, 215 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 16 - protein, 209 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 17 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 18 - protein, 174 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 19 - protein, 171 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 20 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKD

SEQ ID NO: 21 - protein, 147 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 22 - protein, 137 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRCDKPRR

SEQ ID NO: 23 - protein, 371 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 24 - protein, 327 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 25 - protein, 395 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 26 - protein, 412 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 27 - protein, 371 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKD

SEQ ID NO: 28 - protein, 389 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 29 - protein, 354 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 30 - protein, 317 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRCDKPRR

(19) United States
(12) Patent Application Publication
(10) Pub. No.: US 2019/0062812 A1
(43) Pub. Date: Feb. 28, 2019

(54) TB BIOMARKERS

(71) Applicants: Medical Research Council, Swindon (GB); Imperial Innovations Limited, London (GB)

(72) Inventors: Beate KAMPMANN, London (GB); Toyin TOGUN, Montreal (CA); Clive HOGGART, London (GB)

(73) Assignees: Medical Research Council, Swindon (GB); Imperial Innovations Limited, London (GB)

(21) Appl. No.: 16/074,675

(22) PCT Filed: Feb. 3, 2017
(86) PCT No.: PCT/GB2017/050272

§ 371 Date: Aug. 1, 2018

(30) Foreign Application Priority Data
Feb. 9, 2016 (GB) 1602305.3

(51) Int. Cl. C12Q 1/689; A61K 31/4409; A61K 31/496; A61K 31/4965; A61K 31/133; G01N 33/569

(52) U.S. Cl. CPC C12Q 1/689; A61K 31/4409; A61K 31/496; C12Q 2600/158; A61K 31/133; G01N 33/5695; A61K 31/4965

(57) ABSTRACT

The invention relates to a method for the diagnosis of TB in a subject, the method comprising (a) providing a sample from said subject, said sample being selected from the group consisting of: blood, serum and plasma; (b) determining the concentration in said sample of the following biomarkers: IL-1ra, IL6, IL-7, IL-8, IL-12p70, FGF-basic, IP-10, and VEGF; (c) converting each biomarker concentration determined in (b) into a decile value; and (d) converting each decile value into a binary presence or absence by comparing the decile values of (c) to the following specific quantile cut off values wherein a decile value matching or exceeding the specific quantile cut-off value is converted into the binary presence of the biomarker, and a decile value lower than the specific quantile cut-off value is converted into the binary absence of the biomarker; wherein detecting the presence of each of said biomarkers indicates that the subject has TB. The invention also relates to uses, kits and devices.

### FIELD OF THE INVENTION

The invention relates to detection of TB, in particular childhood TB.

### BACKGROUND TO THE INVENTION

Detection of TB is a problem, particularly in children. The current gold standard involves bacteriological assessment. However, sputum samples can be difficult to obtain. Even when successfully obtained, sputum samples from children can exhibit a paucity of bacilli, making direct detection in the sample difficult or impossible. In these circumstances, culture must be carried out in order for the low number of bacilli in the sample to expand to detectable levels. This is laborious and costly and requires specialised laboratory resources, which are drawbacks. More importantly, culture still lacks sensitivity in children. Moreover, culture takes approximately six weeks and this introduces a clinically significant delay to the obtaining of the diagnosis which is a serious problem for patient outcomes. In addition, it is particularly difficult to obtain sputum samples from children, both in practice and in volume. Placing patients, especially children, on speculative treatment without a definitive diagnosis is a serious cost burden as well as the medical risks and complications which such a step would entail.

Dhanasekaran et al 2013 (Genes and Immunity vol 14 pages 356-364) describe identification of biomarkers for Mycobacterium tuberculosis (M.tb.) infection and disease in BCG-vaccinated young children in Southern India. A combination of 11 biomarkers is described with only moderate discriminatory power. Unstimulated whole blood supernatants did not identify cytokine expression differences (pages 359-360).

WO2014/020343 (Proteinlogic Ltd) discloses biomarkers for diagnosing and/or monitoring tuberculosis. This document is focussed on adults. Childhood TB is mentioned only once, and the age of the subjects is not specified. The only exemplification is confined to adults.

Kumar et al 2013 (Clinical and Vaccine Immunology vol 20 pages 704-711) discloses circulating biomarkers of pulmonary and extrapulmonary tuberculosis in children. It is disclosed that paediatric TB was associated with elevated plasma TGF-beta, IL-21 and IL-23 levels. It is disclosed that no significant differences were found for cytokines, for most type 17 and type 1 interferons, or most cytokines associated with immune modulation.

Hur et al 2015 (Journal of Infection vol 70 pages 346-355) disclose adjunctive biomarkers for improving diagnosis of tuberculosis and monitoring therapeutic effects. VEGF is discussed. No disclosure of childhood TB.

Serene et al 2012 (Biomarkers vol 17 pages 1-8) disclose host biomarkers of clinical relevance in tuberculosis: review of gene and protein expression studies. IL-6, IL-22 and IP-10 were mentioned. No disclosure of childhood TB.

Sutherland et al 2012 (PLoS ONE vol 7 epub number: e30324) disclose highly accurate diagnosis of pleural tuberculosis by immunological analysis of the pleural effusion. IL-6 and IP-10 are mentioned. The work is focussed on adults. The work is focussed on pleural fluid.

WO2015/040377 (Medical Research Council) discloses biomarkers for tuberculosis. IL-1ra, FGF and VEGF are mentioned. The work is focussed on adults. The work is focussed on sputum.

The current gold standard for diagnosis is direct detection of the infectious pathogen Mycobacterium tuberculosis (M.tb.) in a clinical specimen such as sputum.

Differentiating active tuberculosis (TB) disease from other respiratory tract infections (OD) constitutes a major challenge in the management of children with suspected intrathoracic TB disease.

The present invention seeks to overcome problem(s) associated with the prior art.

### SUMMARY OF THE INVENTION

Prior art methods have been based on analysis of sputum, or have been based on extraction and manipulation (such as stimulation) of white blood cells. By contrast, the present inventors have based their detection on indicators which can be found directly in samples taken from the patient. In particular, the inventors have based their detection on a blood sample, and direct detection of markers present in that blood sample. This has the advantage of lending itself to development of a point of care test. This has the advantage of avoiding manipulations and stimulations which are part of the prior art techniques. In particular, the invention is advantageously based on unstimulated blood supernatant.

Thus in one aspect the invention provides a method for the diagnosis of TB in a subject, the method comprising;
(a) providing a sample from said subject, said sample being selected from the group consisting of: blood, serum and plasma;
(b) determining the concentration in said sample of the following biomarkers: IL-1ra, IL6, IL-7, IL-8. IL-12p70, FGF-basic, IP-10, and VEGF;
(c) converting each biomarker concentration determined in (b) into a decile value; and
(d) converting each decile value into a binary presence or absence by comparing the decile values of (c) to the following specific quantile cut-off values:

| Biomarker | Specific quantile cut-off value |
|---|---|
| IL-1ra | 3 |
| IL6 | 6 |
| IL-7 | 8 |
| IL-8 | 9 |
| IL-12p70 | 9 |
| FGF-basic | 3 |
| IP-10 | 4 |
| VEGF | 9 |

wherein a decile value matching or exceeding the specific quantile cut-off value is converted into the binary presence of the biomarker, and a decile value lower than the specific quantile cut-off value is converted into the binary absence of the biomarker;

wherein detecting the presence of each of said biomarkers indicates that the subject has TB.

Suitably step (c) converting each concentration determined in (b) into a decile value comprises the steps of:

(ci) comparing the concentration of each biomarker determined in (b) to a reference frequency distribution of concentrations of said biomarker; and

(cii) reading out the decile value from the frequency distribution for the concentration of said biomarker.

Suitably step (c) converting each concentration determined in (b) into a decile value comprises the steps of:

(ci) comparing the concentration of each biomarker determined in (b) to a kernel density estimate of concentrations of said biomarker; and

(cii) reading out the decile value from the kernel density estimate for the concentration of said biomarker.

Suitably the reference frequency distribution or kernel density estimate is generated by measuring the concentration of the biomarker in a number of subjects, for example a minimum of 100 subjects, and compiling those measurements into a frequency distribution/kernel density estimate. Alternatively the frequency distributions (kernel density estimates) presented in FIGS. 2 to 9 herein may be used. In this embodiment step (c) converting each concentration determined in (b) into a decile value comprises the steps of:

(ci) comparing the concentration of each biomarker determined in (b) to the corresponding reference frequency distribution/kernel density estimate of concentrations of said biomarker selected from FIGS. 2 to 9; and

(cii) reading out the decile value from the frequency distribution/kernel density estimate for the concentration of said biomarker.

Suitably determining the concentration of each biomarker comprises:

(bi) detection by contacting the sample with an antibody or antigen binding fragment thereof capable of specifically binding the biomarker; and

(bii) quantification of said binding.

Suitably determining the concentration of each biomarker comprises detection of the mRNA for the biomarker, wherein detection of the mRNA comprises:

(bi) contacting the sample with specific nucleic acid probe(s) or primer(s) for the biomarker; and

(bii) quantification of said probe(s) or primer(s).

Suitably said probe or primer is a non-naturally occurring nucleic acid sequence.

Suitably said probe or primer is an artificial or man-made molecule. Suitably said probe or primer is isolated and/or purified. Suitably said probe or primer comprises single stranded nucleic acid. Suitably said probe or primer comprises a label moiety attached thereto. Suitably said label is covalently attached. Suitably said label may be a fluorescent or radioactive label or a Qdot, nanocrystal or nanoparticle, most suitably a fluorescent label.

Suitably said sample is a sample of serum or plasma.

Suitably said serum or plasma is essentially cell free.

Suitably the subject is 16 years old or younger, preferably 15 years old or younger. Suitably the subject is 2 years old or older, preferably 5 years old or older. Suitably the subject is 5 to 15 years old.

In one embodiment, suitably said method further comprises determining the concentration in said sample of biomarker EC-stimulated VEGF (specific quantile cut-off value 2).

In one aspect, the invention relates to a kit comprising reagent(s) for the specific detection of each of the following biomarkers: IL-1ra, IL6, IL-7, IL-8, IL-12p70, FGF-basic, IP-10, and VEGF.

In one aspect, the invention relates to a kit comprising reagents for the specific detection of mRNA encoding each of the following biomarkers: IL-1ra, IL6, IL-7, IL-8, IL-12p70, FGF-basic, IP-10, and VEGF.

Suitably said reagents each comprise an antibody or antigen binding fragment thereof selected from the group consisting of a Fab fragment, a Fab′ fragment, a F(ab′)2 fragment, a scFv, aFv, a rIgG, and a diabody.

In one aspect, the invention relates to a device comprising an array of materials which together are capable of specifically binding each of the following biomarkers: IL-1ra, IL6, IL-7, IL-8. IL-12p70, FGF-basic. IP-10, and VEGF, each material within the array being capable of specifically binding one of said biomarkers.

In one aspect, the invention relates to a device comprising an array of materials which together are capable of detecting mRNA specific for each of the following biomarkers: IL-1ra, IL6, IL-7, IL-8, IL-12p70, FGF-basic, IP-10, and VEGF, each material within the array being capable of specifically detecting one of said mRNAs.

Suitably said device is a lateral flow device.

In one aspect, the invention relates to a method of treating a subject comprising carrying out the method as described above, wherein if it is determined that the subject has TB, a regimen of 2HRZE/4HR (2 months HRZE followed by 4 months HR wherein H=isoniazid, R=rifampicin, Z=pyrazinamide, E=ethambutol) is administered to said subject.

In one aspect, the invention relates to use of HRZE wherein H=isoniazid, R=rifampicin, Z=pyrazinamide, E=ethambutol for treatment of TB in a subject, wherein the method as described above is carried out for said subject, wherein if it is determined that the subject has TB then HRZE is administered to said subject for two months and then HR is administered to said subject for four months. Suitably a treatment regimen of dosing said subject at least three times per week during the first two months is selected, preferably a treatment regimen of dosing said subject daily during the first two months is selected.

In one aspect, the invention relates to tablets of H 75 mg+R 150 mg+Z400 mg+E 275 mg for treatment of TB in a subject, wherein the method as described above is carried out for said subject, wherein if it is determined that the subject has TB then HRZE is administered to said subject for two months, followed by 3 tablets of H 75 mg+1.5 tablets of R 150 mg for four months.

In one aspect, the invention relates to a process for selecting a treatment regimen, said process comprising

carrying out the method as described above, wherein if it is determined that the subject has TB then a treatment regimen of 2HRZE/4HR (2 months HRZE followed by 4 months HR wherein H=isoniazid, R=rifampicin, Z=pyrazinamide, E=ethambutol) is selected.

In one aspect, the invention relates to use of a combination of materials each of which recognises, specifically binds to or has affinity for one of the following biomarkers: IL-1ra, IL6, IL-7, IL-8, IL-12p70, FGF-basic, IP-10, and VEGF, wherein said combination includes at least one such material for each of said biomarkers, for aiding diagnosis of TB in a subject. Suitably said material comprises an antibody or antigen binding fragment thereof.

In one aspect, the invention relates to use for aiding diagnosis of TB in a subject, of a combination of materials each of which recognises, specifically binds to or has affinity for mRNA of one or more of the following biomarkers: IL-1ra, IL6, IL-7, IL-8, IL-12p70, FGF-basic, IP-10, and VEGF. Suitably said material comprises a nucleic acid primer or probe.

In one aspect, the invention relates to a apparatus comprising logic configured to carry out the method as described above.

In one aspect, the invention relates to a computer program product operable, when executed on a computer, to perform the method steps as described above.

### DETAILED DESCRIPTION OF THE INVENTION

The inventors teach identification of novel host biomarkers for childhood TB. The inventors hypothesised that a unique biosignature for TB disease in children could be identified.

‘TB’ means Tuberculosis; this is a disease caused by the bacterium Mycobacterium tuberculosis (sometimes referred to as MTB).

It has been a challenge in the field of TB to provide a simple diagnostic test. The test needs to be reliable. The test needs to be accurate. The test should ideally involve the minimum of tooling/equipment, especially since TB is often a problem in developing countries where laboratory facilities can be few and/or can be geographically distant from the patients being tested.

### Sample

The sample may be from a subject. The subject is suitably a mammal, most suitably a human.

Suitably the methods do not involve actual collection of the sample. Suitably the sample is an in vitro sample.

Methods of the invention are suitably performed on an isolated sample from the subject being investigated. Thus, suitably the methods are methods which may be conducted in a laboratory setting without the need for the subject to be present. Suitably the methods are carried out in vitro i.e. suitably the methods are in vitro methods. Suitably the methods are extracorporeal methods.

Suitably the invention may be applied to analysis of nucleic adds. Suitably, nucleic acid is prepared from the sample collected from the subject of interest, e.g. by extraction of nucleic acid from white blood cells in the sample. Suitably, the sample comprises nucleic acid. Suitably, the sample consists of nucleic acid. Suitably, the nucleic acid comprises, or is, mRNA or cDNA, suitably mRNA.

Most suitably the invention may be applied to analysis of protein biomarkers. Most suitably proteins in the sample are analysed.

Suitably the sample is an in vitro sample.

Suitably the sample is an extracorporeal sample.

Suitably the sample is blood.

Suitably the sample is blood supernatant.

Suitably the sample is serum. Serum may be obtained as the fluid collected from a blood sample which has been dotted.

Suitably the sample is plasma. Plasma may be obtained as the fluid collected from a blood sample which has been centrifuged to pellet the blood cells present. Alternatively plasma may be obtained by filtration to remove the blood cells present.

Suitably the blood or blood supernatant/serum/plasma is unstimulated.

It is an advantage of the invention that the analysis converts each measured biomarker into a binary output. For example, each cytokine biomarker examined is turned into a binary YES/NO data point. By contrast, existing prior art approaches need quantitative readouts.

It is an advantage of the invention that diagnostic accuracy is achieved at a level comparable to bacteriological culture. It is an advantage of the invention that there are no time delays comparable to those experienced with bacteriological culture.

Prior art techniques use cells, either bacterial cells or isolated blood cells. It is an advantage of the invention that no cells are required to be cultured, and no cells are required to be isolated.

It is an advantage of the invention that the host response/host signature is analysed. Without wishing to be bound by theory, the existing attempts to create a blood test for TB have focussed on challenging or stimulating immune cells from the subject and studying the response. However, in principle this approach is always examining a “recall response”. This is a response derived from “memory” of the immune system which has previously encountered a TB bacterium. Firstly, this requires a stimulation of the sample either with bacteria or with antigens, which is labour intensive and costly. However, more importantly, in principle, this type of approach is only able to assay a secondary response. By contrast, the present invention is concerned with assessment of the primary response. This is especially important and useful when applied to children, since dearly each individual patient is at some point in their lives exposed to TB for the first time. If a method such as a prior art method is only focussed on assessing a secondary response, then it is unlikely ever to be able to detect a response the first time such a subject encounters TB. It is an advantage of the present invention that the direct primary response is being assessed.

Suitably the sample is a cell free sample. Suitably the sample does not comprise cells.

Suitably the cells are removed from the blood sample by centrifugation and retrieval of the supernatant.

Cells may be removed from the sample by any method known in the art. For example filtration.

A key reason for excluding cells from the analysis is because blood is deeply red in colour due to the presence of the erythrocyte red blood cells. Typically the detection step used to assess the presence or absence of the biomarkers is light sensitive. Therefore, advantageously the sample is free of cells in order to avoid being confounded by the red colour of whole blood. In principle, any approach which circumvents the deep red colour of whole blood could be employed. Suitably this may be by cell lysis. More suitably the cells are removed from the blood before analysis. Suitably this is by centrifugation. Suitably this may be by filtration. Suitably this may be by lateral flow.

In some settings, it may be possible to use a whole blood sample in the method of the invention, for example by using lateral flow. In this scenario, the whole blood sample is placed in a lateral flow device. The fluid component such as plasma/serum then migrates, so at the point of assessment it is cell free.

### Lateral Flow Assay and Other Devices

Lateral flows test assays (LFA)—also referred to as lateral flow immunochromatographic assays—require minimal infrastructure and have been used to develop cheap and simple devices for rapid medical diagnosis and screening, point of care tests or laboratory use. The assay is based on the detection of the presence of a specified target analyte in a sample for mostly qualitative and occasional quantitative analysis. Common applications of the LFA include its use in home pregnancy test, monitoring of diabetes and rapid diagnosis of HIV or parasitic and bacterial infections. As discussed extensively in review articles (such as Sajid M, Kawde A, Daud M. Designs, formats and applications of lateral flow assay: A literature review. Journal of Saudi Chemical Society. 2014), a typical LFA strip is made up of four parts:
Sample application pad: This is an absorbent pad made of cellulose or glass fibre on which the sample is applied and its main function is to transport the sample (e.g. blood) containing the analyte ‘downstream’ to the other part of the LFA strip. It may also pre-treat the sample, including separation into components such as plasma, before its transportation.
Conjugate pad: this contains immobilized and labelled antibody that is specific for the target analyte. The antibody is conjugated to coloured particles such as latex or nanometre sized particles and the labelled antibody conjugate is released upon contact with moving liquid sample.
Nitrocellulose or Reaction membrane: This membrane allows movement of complexes generated from the conjugated pad under capillary action. The membrane is further divided into test and control lines.
Adsorbent pad: This pad works as a ‘sink’ at the end of the strip and it is designed to further draw the sample across the reaction by capillary action.

FIG. 10 shows the standard layout of a lateral flow device (Saied Assadollahi, Christiane Reininger, Roland Palkovits, Peter Pointl and Thomas Schalkhammer. ‘From Lateral Flow Devices to a Novel Nano-Color Microfluidic Assay.’ Sensors 2009 (9); 6084-6100; doi:10.3390/s90806084).

Lateral flow assay test can work in two main formats, which are the sandwich and competitive assays. Sandwich LFA are designed for detection of large molecular weight molecules including proteins with multiple antigenic sites (e.g. HIV, hCG), while competitive assays are designed to test small molecules with single epitopes. In sandwich LFA, a positive test will show coloured bands on the test line, while in competitive LFA the test line will show coloured band in negative samples.

### Multiplex Detection Format

In clinical diagnosis, the higher specificity or predictive accuracy of multiple inter-dependent analytes that are detected simultaneously under the same condition has led to the development of multiplex LFA detection format used for the detection of more than one target analyte (Panhotra B R, Hassan Z U, Joshi C S, Bahrani A. Visual detection of multiple viral amplicons by dipstick assay: its application in screening of blood donors a welcome tool for the limited resource settings. Journal of clinical microbiology. 2005 December; 43(12):6218; author reply-9. Pub Med PMID: 16333138. Pubmed Central PMCID: 1317223; Corstjens P L, de Dood C J, van der Ploeg-van Schip J J, Wiesmeijer K C, Riuttamaki T, van Meijgaarden K E, et al. Lateral flow assay for simultaneous detection of cellular- and humoral immune responses. Clinical biochemistry. 2011 October; 44(14-15):1241-6. Pub Med PMID: 21763300. Pubmed Central PMCID: 3177995). In this format, the assay is performed over a strip with number of test lines equal to the number of target analytes as recently described for the detection of four common human papillomavirus (HPV) types (Xu Y, Liu Y, Wu Y, XiaX, Liao Y, Li Q. Fluorescent probe-based lateral flow assay for multiplex nucleic acid detection. Analytical chemistry. 2014 Jun. 17; 86(12):5611-4. Pub Med PMID: 24892496).

FIG. 11 shows multiplex detection format of LFA (Ye Xu, Yinghua Liu, Yan Wu, Xiaohu Xia, Yiqun Liao and Qingge Li. ‘Flourescent Probe-Based Lateral Flow Assay for multiplex Nucleic Acid Detection.’ Anal Chem., 2014, 86(12), 5611-5614).

### Clinical Applications

Lateral Flow Assays have found important use in clinical diagnosis and as point of care tests through the detection of clinical analytes in plasma, serum, urine and other clinical samples. A ready example is the home pregnancy testing kits. In tuberculosis (TB), the urinary lipoarabinomannan (LAM) test is a LFA test with low sensitivity (52-59% but a high specificity (>94%) (Lawn S D, Dheda K, Kerkhoff A D, Peter J G, Dorman S, Boehme C C, et al. Determine TB-LAM lateral flow urine antigen assay for HIV-associated tuberculosis: recommendations on the design and reporting of clinical studies. BMC infectious diseases. 2013; 13:407. Pub Med PMID: 24004840. Pubmed Central PMCID: 3846798). The inventors also recently reported that the use of fluorescent up-converting phosphor (UCP) reporter technology combined with LFA to detect IP-10 and CCL4 simultaneously on the same strip has potential to be developed as a point of care test for pleural TB (Sutherland J S, Mendy J F, Gindeh A, Walzl G, Togun T, Owolabi O, et al. Use of lateral flow assays to determine IP-10 and CCL4 levels in pleural effusions and whole blood for TB diagnosis. Tuberculosis (Edinb). 2015). A multi-centre study in Africa similarly reported the applicability of the low-tech and robust UCP-LFA platform as a convenient quantitative assay for detection of multiple chemokines in whole blood (Corstjens P L, Tjon Kon Fat E M, de Dood C J, van der Ploeg-van Schip J J, Franken K L, Chegou N N, et al. Multi-center evaluation of a user-friendly lateral flow assay to determine IP-10 and CCL4 levels in blood of TB and non-TB cases in Africa. Clinical biochemistry. 2015 Aug. 15. Pub Med PMID: 26285074).

Therefore, the invention relates to advice comprising an array of materials which together are capable of binding each of the following biomarkers: IL-1ra, IL6, IL-7, IL-8, IL-12p70, FGF-basic, IP-10, and VEGF, each material within the array being capable of binding one of said biomarkers, wherein said device is a lateral flow device. In this embodiment, the array of materials suitably comprises a number of test lines equal to the number of biomarkers. Suitably each test line comprises material capable of binding one such biomarker. Suitably each test line comprises material capable of binding a different such biomarker.

Alternatively, if the device comprises a ‘chip’ or ‘biochip’, the array may comprise a spatial arrangement of the materials such as a grid or other defined arrangement such as a geometric pattern.

Suitably each material capable of binding one of said biomarkers is immobilised within the device.

Suitably each material capable of binding one of said biomarkers is modified.

Suitably each material capable of binding one of said biomarkers is labelled. Suitably the label is covalently attached. Suitably the label is a dye.

Suitably each material capable of binding one of said biomarkers is a different antibody or antigen binding fragment thereof, wherein the antigen binding fragment thereof is selected from the group consisting of a Fab fragment, a Fab′ fragment, a F(ab′)2 fragment, a scFv, a Fv, a rIgG, and a diabody.

Suitably the antibody or antigen binding fragment thereof is a non-human antibody or antigen binding fragment thereof. Suitably the antibody or antigen binding fragment thereof is recombinant, e.g. made by in vitro expression of a recombinant nucleic aid sequence. Suitably the antibody or antigen binding fragment thereof is purified and/or isolated.

Non-antibody reagents for detection e.g. of protein biomarkers as described above may also be used, (e.g. for detection in the methods, as material in the devices, as reagents in the kits, or other applications of the invention) for example phage display particles offering specific binding peptides, affimers, aptamers, nucleic acids binding specific protein or peptide sequences, small molecules with specific binding properties or other such specific binding partner(s) of the biomarkers described.

### Signature

Prior art approaches have identified extremely large signatures such as requiring the analysis of 50 or more individual biomarkers. It is an advantage of the invention that the signature requires analysis of only 8 biomarkers.

Prior art attempts have involved flow cytometry. However, flow cytometry is extremely labour intensive, expensive, and is unsuitable for the provision of a bedside/point of care test.

Prior art approaches have involved analysis of the transcriptome (i.e. of mRNAs). However, these approaches have typically also involved assessment of at least 50 genes, which is a problem.

Data is presented in the examples section in support of the AUC/specificity/sensitivity of the methods of the invention.

Most importantly, the positive and negative predictive values are provided. In the field of TB, the predictive values may be regarded as more important even than the sensitivity/specificity of the method. It is an advantage that the invention delivers extremely robust positive and negative predictive values. This is discussed in more detail in the section below entitled “Predictive Values/Applications Of The Method” The inventors undertook a large and complex analysis involving numerous intellectual choices in arriving at the 8 biomarker signature. In particular, it is important to note that this particular signature has special properties. For example, it is significantly different from a 7 biomarkers signature, which is unsuitable. Analysis of the signature attempting to drop any of the 8 biomarkers disclosed leads to a drop in specificity of approximately 50% and/or a drop in predictive value of approximately 25%. These figures clearly illustrate that the teaching of an 8 biomarker signature according to the invention is not merely an iterative or arbitrary choice but presents clinically useful information which is a step change from that obtained with even one fewer marker. It is surprising that such a sharp and dramatic effect can be observed in this manner.

Suitably the signature comprises 8 biomarkers.

Suitably the biomarkers are as set out in the table below.

### REFERENCE SEQUENCES

Suitably the reference sequences of the biomarkers of interest are as defined in the following table:

| | | | | | mRNA |
|---|---|---|---|---|---|
| | Protein | | | | Accession |
| | Accession | | | | Number |
| | Number | Canonical | Protein | Gene | (RefSeq |
| Biomarker | (UniProtKB) | sequence | Isoforms | name | Release 73) |
| IL-1ra | P18510 | P18510-1 | P18510-2 | IL1RN | NM_173841.2 |
| | | | P18510-3 | | NM_000577.4 |
| | | | P18510-4 | | NM_173842.2 |
| | | | | | NM_173843.2 |
| IL6 | P05231 | P05231-1 | | IL6 | NM_000600.3 |
| IL-7 | P13232 | P13232-1 | P13232-2 | IL7 | NM_000880.1 |
| | | | P13232-3 | | NM_001199886.1 |
| | | | | | NM_001199887.1 |
| | | | | | NM_001199888.1 |
| IL-8 | P10145 | P10145-1 | P10145-2 | CXCL8 | NM_000584.3 |
| IL-12p70 | P29460 | P29460-1 | | IL12B | NM_002187.2 |
| FGF-basic | P09038 | P09038-4 | P09038-1 | FGF2 | NM_002006.4 |
| | | | P09038-2 | | |
| | | | P09038-3 | | |
| IP-10 | P02778 | P02778-1 | | CXCL10 | NM_001565.3 |
| VEGF | P15692 | P15692-1 | P15692-2 | VEGFA | NM_001025366.2 |
| | | | P15692-3 | | NM_001025367.2 |
| | | | P15692-4 | | NM_001025368.2 |
| | | | P15692-5 | | NM_001025369.2 |
| | | | P15692-6 | | NM_001025370.2 |
| | | | P15692-8 | | NM_001033756.2 |
| | | | P15692-9 | | NM_001171622.1 |
| | | | P15692-10 | | NM_001171623.1 |
| | | | P15692-11 | | NM_001171624.1 |
| | | | P15692-12 | | NM_001171625.1 |
| | | | P15692-13 | | NM_001171626.1 |
| | | | P15692-14 | | NM_001171627.1 |
| | | | P15692-15 | | NM_001171628.1 |
| | | | P15692-16 | | NM_001171629.1 |
| | | | P15692-17 | | NM_001171630.1 |
| | | | P15692-18 | | NM_001204384.1 |
| | | | | | NM_001204385.1 |
| | | | | | NM_001287044.1 |
| | | | | | NM_001317010.1 |
| | | | | | NM_003376.5 |

The sequences are hereby incorporated herein by reference.

The skilled worker only has to identify the correct gene/protein being used in the analysis. The guidance provided is not intended to restrict the invention rigidly to the specific single exemplary sequences provided. Gene sequences (and therefore protein sequences) are known to vary between individuals e.g. due to allelic variance or mutations between individuals. The information provided is to assist the operator in working the invention by assaying the correct gene. Ultimately the gene product (such as mRNA or more suitably protein) is actually assayed. Therefore minor or minimal allelic or mutational differences between individuals are not important, what is important is that the correct gene (gene product) is assayed using the guidance provided.

Suitably where the biomarker name is used, this means the corresponding amino acid or nucleic acid sequence from the above table. Suitably for amino acid sequences, the canonical sequence is preferred. Suitably for nucleic acid sequences, the most recent (e.g. highest numbered) nucleic acid sequence is preferred.

It will be understood that the invention may equally make use of detection of fragment(s), variant(s) or mutant(s) of these biomarkers. Suitably any such fragment(s), variant(s) or mutant(s) have at least 80% sequence identity to the reference sequences along the whole length of said fragment(s), variant(s) or mutant(s), suitably 90%, suitably 95%, suitably 98% sequence identity along the whole length of said fragment(s), variant(s) or mutant(s).

Database Release

Sequences deposited in databases can changeover time. Suitably the current version of sequence database(s) are relied upon. Alternatively, the release in force at the date of filing is relied upon.

As the skilled person knows, the accession numbers may be version/dated accession numbers. The citeable accession numbers for the current database entry are the same as above, but omitting the decimal point and any subsequent digits e.g. for VEGF a version/dated accession number is P15692-18; the current entry is obtained using P15692 and so on.

GenBank is the NIH genetic sequence database, an annotated collection of all publicly available DNA sequences (National Center for Biotechnology Information, U.S. National Library of Medicine 8600 Rockville Pike. Bethesda Md., 20894 USA; Nucleic Acids Research, 2013 January; 41(D1):D36-42) and accession numbers provided relate to this unless otherwise apparent. Suitably the GenBank database release referred to is 15 Oct. 2015, NCBI-GenBank Release210.0.

UniProt (Universal Protein Resource) is a comprehensive catalogue of information on proteins (‘UniProt: a hub for protein information’ Nucleic Acids Res. 43: D204-D212 (2015).). For the avoidance of doubt, UniProt Release 2015_11 is relied upon.

In more detail, the UniProt consortium European Bioinformatics Institute (EBI), SIB Swiss Institute of Bioinformatics and Protein Information Resource (PIR)'s UniProt Knowledgebase (UniProtKB) Release 2015_11 (11 Nov. 2015) is relied upon.

Treatments

It should be noted that treatment for TB is a minimum six month program of drugs. This is expensive and can be very demanding on the patient. Dosage has to be very regular, such as multiple doses per week and ideally daily, which is a heavy burden on the healthcare provider as well as the patient. Therefore, it is a problem in the art to avoid the mistreatment of patients i.e. the mis-prescription of TB drugs to a patient who does not in fact have TB. The present invention alleviates this problem by providing a robust tool for diagnosis (or for aiding diagnosis).

If it is decided in view of the method of the invention that the subject has TB, then the physician should prescribe the treatment for TB. In one embodiment the invention provides a method of treating a patient comprising determining if they have TB according to the method(s) disclosed herein, wherein if the patient is determined to have TB then the treatment for TB is prescribed, or more suitably administered. In another embodiment the methods of the invention are used to aid diagnosis of a patient who need not be present during the diagnostic step in the strict sense (i.e. in this embodiment the invention is not directed at diagnosis for curative purposes stricto sensu); the physician may then take the information provided by the invention into account when diagnosing and/or planning the treatment for said subject.

Thus it is an advantage of the invention that unnecessary drugs can be avoided. In one embodiment, the test of the invention finds application as a rule-in test rather than a rule-out test. In other words, if the method(s) of the invention are used to determine that a subject has TB, they should definitely be treated for TB. In the alternative, if the methods of the invention are used and it is not determined that a subject has TB, then further investigation may be useful—a subject is suitably not ‘ruled out’ from possibly having TB if the methods of the invention do not determine that they have TB. In terms of a ‘rule-in’ test, the methods of the invention have a comparable performance to the gold standard in the art (bacterial culture). Thus it is an advantage of the invention that a positive finding using the methods of the invention provides a very high level of confidence that the subject has TB and should be treated for TB. Suitably TB treatment is as recommended by the World Health Organisation (WHO). The WHO may amend its guidelines from time to time—suitably treatment is as per the guidelines at the date of working the invention to treat a subject. More suitably this treatment is as per the guidelines at the filing date of this document. If any further guidance is needed, most suitably treatment is as per the guidelines in the WHO 2010 publication “Guidelines for national programmes, fourth edition” ISBN: 9789241547833 (e.g. http://www.who.int/tb/publications/9789241547833/en/). This document is hereby incorporated herein by reference, specifically for the teaching of the specific treatment regime for TB.

Exemplary treatments are set out below.

Suitably TB treatment comprises the standard 6-month course of 4 anti microbial drugs as recommended by the WHO.

Suitably TB treatment comprises 6 months of rifampicin.

Suitably TB treatment comprises six (6) months treatment with specific anti-TB drugs (Isoniazid, Rifampicin, Pyrazinamide and Ethambutol for first 2 months, followed by 4 months of Isoniazid and Rifampicin only).

Suitably patients with TB may receive a daily intensive phase followed by a three times weekly continuation phase [2HRZE/4(HR)3]; suitably each dose is directly observed. Three times weekly dosing throughout therapy [2(HRZE)3/4(HR)3] may be used as an alternative, provided that every dose is directly observed and the patient is NOT living with HIV or living in an HIV-prevalent setting.

Suitably the dosing is not less than 3 times per week. Suitably the dosing is 3 times per week or more. Most suitably the dosing is daily.

Most suitably the dosing frequency for patients with TB is daily throughout the course of therapy [2HRZE/4HR].

Suitably if the patient is living with HIV or living in an HIV-prevalent setting the dosing is daily throughout the course of therapy.

Summary of WHO treatment guidance (WHO 2010 publication “Guidelines for national programmes, fourth edition” page 5):

TABLE A
STANDARD REGIMEN AND DOSING FREQUENCY FOR NEW TB PATIENTS

| Intensive phase | Continuation phase | Comments |
|---|---|---|
| 2 months of HRZE^{a} | 4 months of HR | |
| 2 months of HRZE | 4 months of HRE | Applies only in countries with high levels of |
| | | isoniazid resistance in new TB patients, and |
| | | where isoniazid drug susceptibility testing |
| | | in new patients is not done (or results are |
| | | unavailable) before the continuation phase |
| | | begins |
| Dosing frequency | | |
| Daily | Daily | Optimal |
| Daily | 3 times per week | Acceptable alternative for any new TB patient |
| | | receiving directly observed therapy |
| 3 times per week | 3 times per week | Acceptable alternative provided that the |
| | | patient is receiving directly observed therapy |
| | | and is NOT living with HIV or living in an HIV- |
| | | prevalent setting (see Chapter 5) |

^{a}WHO no longer recommends omission of ethambutol during the intensive phase of treatment for patients with non-cavitary, smear-negative pulmonary TB or extrapulmonary disease who are known to be HIV-negative.
Note:
Daily (rather than three times weekly) intensive-phase dosing may help to prevent acquired drug resistance in TB patients starting treatment with Isoniazid resistance (see Annex 2).

### Abbreviations Used

H=isoniazid,
R=rifampicin,
Z=pyrazinamide,
E=ethambutol,
S=streptomycin.

The standard treatment above is suitably not applied to muti drug resistant TB (MDR TB). Suitably drug susceptibility testing is undertaken before treatment, in accordance with WHO guidelines. If treating a TB patient whose treatment has failed or other patient with high likelihood of multidrug-resistant TB (MDR-TB), suitably treatment should be started on an empirical MDR regimen as recommended by WHO. Most suitably the invention is applied to ‘new’ TB, i.e. new patients tested according to the invention.

Further or additional treatment may depend on whichever diagnosis is made; usually a course of antibiotics if it is a bacterial respiratory tract infection.

Administration may be by tablet or by injection such as intramuscular injection. For HRZE/HR regimes, suitably administration is by tablet.

Typical tablets comprise the following doses:

H 75 mg+R 150 mg+Z 400 mg+E 275 mg tablets.

Subjects are administered or prescribed a number of tablets according to their body weight (and/or any other relevant factors if necessary) to achieve the correct dose. This number may include fractions of a tablet. A typical dose for a subject of 30-39 Kg body weight is 2 tablets of H 75 mg+R 150 mg+Z 400 mg+E 275 mg daily during the first 2 months of treatment, followed by 1.5 tablets of H 150 mg+R 150 mg during months 3 to 6 of treatment. The determination of exact dose e.g. based on body weight is a matter for the physician.

Statistical Analysis

The inventors considered standard approaches to statistical analysis. However, the inventors had insight that standard regression models tend to lead to over optimism. This is particularly true considering the multidimensionality of the data arising from the large number of cytokine covariates some of which are highly correlated with each other. For various reasons, the inventors used a different approach, and simultaneously tried to shrink the marker set (i.e. to reduce the dimensionality) whilst applying penalties for shrinkage in the statistical analysis. Without wishing to be bound by theory, their reasoning was that if a signature assesses two markers which move in the same direction, then qualitatively the same information is being obtained from two comparable sources. This provides an opportunity to eliminate one of those markers, thereby simplifying the signature without compromising the quality of information obtained. The corollary of this is that if two analyses are providing information in two different directions, then this can be seen to provide extra information to improve the signature. In this manner, the inventors sought to remove any markers which might be considered as statistically related to one another, thereby arriving at an improved empirical (reduced) signature but which still delivered excellent diagnostic characteristics.

Marker Selection

The inventors undertook unbiased marker selection. Prior art based approaches have tended to use a “case control” approach. In brief, this might be characterised by settling on TB as a subject to be addressed, selecting healthy patients, selecting patients having TB, and comparing the healthy patients with the TB diseased patients. By contrast, the inventors designed a case-control study nested within a prospective cohort (i.e. nested case-control) approach. They selected a cohort of children using the same selection process to identify children in different geographical locations. Only then did they identify within those cohorts patients having TB and patients not having TB. More importantly, the inventors chose to compare TB to OD (i.e. “other respiratory disease but not TB”). This is because it is a key clinical decision to identify those patients with TB compared to those presenting with other diseases which are not TB. Thus, it can be considered a problem in the art to differentiate TB patients from “other disease” patients.

Another drawback with prior art studies is that they have tended to settle on biomarkers such as cytokines which have been published or associated with TB. By contrast, the inventors took an entirely unbiased approach and did not pick biomarkers by association with TB. They undertook a blind analysis and arrived at the signature of 8 biomarkers without knowing what those individual biomarkers were. Only then did the inventors analyse the identities of the biomarkers in their signature. One example of the surprise of this approach is by considering IFN-γ. The view in the art is that IFN-γ is involved with TB. Indeed, prior art approaches have tried to use IFN-γ and/or IP-10 as biomarkers for TB. It is extremely surprising that the biomarker signature taught herein does not comprise IFN-γ.

Quantiferon (‘QFT’-available commercially from Qiagen Inc.) is an interferon-gamma (IFN-γ) release assay, commonly known as an IGRA, and is a modern alternative to the tuberculin skin test (TST or Mantoux). In overview, QFT measures the cell-mediated immune response (cytokines) to very specific TB antigens. The test is performed by collecting whole blood (1 mL) into each of three blood collection tubes. When the blood of an infected patient is stimulated with the M. tuberculosis-specific antigens in QFT, their T-Cells respond by secreting a cytokine called IFN-γ. The IFN-γ concentration in the plasma is determined using a sensitive ELISA.

Thus, Quantiferon (‘QFT’) is a commercial assay that still measures IFN-gamma following stimulation of blood with antigens specific for M.tb. Some IP-10 assays have also been investigated in the prior art. However, while IP-10 is reportedly more robust than IFN-gamma (i.e. released at a much higher level following stimulation), on its own it cannot distinguish between TB disease and TB sensitization similar to IFN-gamma, which is a problem in the art. Advantageously, in the present invention, what is taught is a combination of markers, which when used together—not individually—can distinguish TB from OD. This is based on the inventors' insight that, given the complexity of TB disease, a combination of markers rather than a single marker has a higher power and specificity to distinguish TB from TB sensitization and/or other disease.

In more detail, the inventors took an unusual approach by analysing various cytokines and chemokines and turning the values of each into deciles. Starting with 27 cytokines/chemokines as candidates, each was transformed into 10 deciles giving 270 for each patient stimulated and 270 for each patient unstimulated.

This represents a categorical (rather than continuous) approach which is itself a key part of the innovative approach taken. This approach has never before been applied to cytokine analysis. This has advantages which include removing the confounding effect of bias or selection in the biomarkers which are used.

The panel of 27 initial candidates did not have any previous association with TB. For example, they were not TB specific. At most, they may be regarded as an “immune panel”. They are simply markers involved in lymphocytes as part of a commercial kit which is not in any way marketed or directed towards TB. To illustrate how surprising the findings were, even the inventors did not predict what they would find by using this approach.

In one embodiment the analysis may be carried out as follows:

frequency distribution>10 deciles>make each a binary quantile.

In one embodiment the analysis may be carried out as follows:

frequency distribution>deciles>10−equal sized quantiles>use each quantile as cut-off to generate a binary variable.

It should be noted that each cytokine may be present across a different range of concentrations so that each of the deciles may not be the same between individual biomarkers. However, the distribution of each individual biomarker is suitably divided into 10 deciles (i.e. 10 equal-sized quantiles) according to its own range of concentrations when present; each biomarker value determined for a patient is then converted into a decile from that frequency distribution.

It should be noted that the invention is directed at obtaining a clinical decision whether a subject has TB or OD.

It is possible to use the invention as a screening tool such as a population screening test.

It is an advantage of the invention that it is helpful in assisting the clinical decision whether a patient has TB or OD.

### Decile Determination

Deciles are generated according to standard statistical techniques. Generation of deciles is a mathematical frequentist procedure that can be derived or generated by any statistical software. A specific variable (e.g. a measured cytokine) with quantitative values when measured from several subjects will have a frequency distribution that is then used to generate the deciles made up of 10 equal-sized quantiles.

In case any further guidance is needed, most suitably deciles are generated as described in the examples section below.

### Subjects

The present invention may be applied to any subject from newborn onwards.

The present invention may be applied to adults or children.

Suitably the subject is 18 years or younger, suitably 16 years or younger, suitably 15 years or younger, suitably 7 years or younger, suitably 6 years or younger, suitably 5 years or younger, suitably 2 years or younger.

It should be noted that most immune systems function as“adult” from 2 to 5 years onwards. Thus, suitably the subject is at least 2 to 5 years old. Suitably the subject is at least 2 years old. Suitably the subject is at least 3 years, suitably at least 4 years, most suitably at least 5 years old.

Suitably the subject is a child.

Suitably the subject is 16 years or younger.

Suitably the subject is 15 years or younger.

Suitably the subject is 2 to 16 years old, suitably 3 to 16, suitably 4 to 16, suitably 5 to 16 years old.

Suitably the subject is 2 to 15 years old, suitably 3 to 15, suitably 4 to 15, suitably 5 to 15 years old.

Suitably the subject to be tested has presented with at least one of the following symptoms: coughing, weight loss, sweating, swollen glands, and optionally sepsis.

The invention may be applied to intra-thoracic TB.

The invention may be applied to pulmonary TB.

The invention may be applied to extra-pulmonary TB.

The invention may be applied to patients which are uninfected with HIV.

The invention may be applied to patients which are infected with HIV.

It should be noted that since the method is based on the immune response, that any subject with a CD4 count of 50 or fewer is unlikely to show a response. Thus, suitably the subject has a CD4 count of 51 or greater. For reference, a normal CD4 count in a healthy human is approximately 1000.

Detection

Suitably the biomarkers described herein are detected by the suitable means in the art. For example, the biomarkers may be detected by one or more antibodies which specifically recognise said biomarkers.

For example, the biomarkers may be detected by an antibody or antigen binding fragment thereof as described above, wherein the antigen binding fragment thereof is selected from the group consisting of a Fab fragment, a Fab′ fragment, a F(ab′)2 fragment, a scFv, a Fv, a rIgG, and a diabody.

The mode of assessing binding of an antibody or antigen binding fragment thereof to detect the markers is a matter of operator choice. In case any guidance is needed, ELISA's could be used for each of the cytokines identified, for example one ELISA for each biomarker. Below are examples of suitable ELI SA reagents.

ELI SA's for the cytokines included in the signature of the invention may be obtained commercially from the following companies, with exemplary product names/details where appropriate:

Quantikine sandwich Elisa from R&D Systems UK, 19 Barton Lane, Abingdon Science Park, Abingdon, OX14 3NB, United Kingdom (Tel +44 (0)800 3734 15).

Human Platinum Elisa or high sensitivity Elisa from eBioscience, Ltd. (Ireland, United Kingdom), 2nd Floor, Titan Court, 3 Bishop Square, Hatfield, AL10 9NA, United Kingdom.

BD OptEIA kits from BD Biosciences, Edmund Halley Road, Oxford Science Park, Oxford, OX4 4DQ (Tel.: +44 1865 781 666; Fax: +44 1865 781 627).

As the skilled worker will be aware, individual ELISAs for each cytokine might be laborious, and/or require larger sample sizes. Therefore it is an advantage to carry out the detection in a multiplex or single sample where possible. This provides advantages such as low volume (particularly important for a paediatric test where lower volumes of blood/serum are desirable), and combination of the cytokines (less labour to complete the test).

There are numerous commercial suppliers of suitable multiplexing kit(s) useful to detect the biomarkers of the signature, for example:

MILLIPLEX MAP Human Cytokine/Chemokine Magnetic Bead Panel—Immunology Multiplex Assay, Catalogue number: HCYTOMAG-60K available from Merck-Millipore, Suite 21, Building 6, Croxley Green Business Park, Watford, Hertfordshire WD18 8YH, United Kingdom.

Human Luminex Performance Assay Base Kit, Panel A [catalogue number LUH000] from R&D Systems UK, 19 Barton Lane, Abingdon Science Park, Abingdon, OX14 3NB, United Kingdom (Tel: +44 (0) 800 3734 15).

Human Cytokine (Chemokine Growth Factor Panel 1 (45 plex), (Catalogue number: EPX450-12171-901) from eBioscience, Ltd., (Ireland, United Kingdom), 2nd Floor, Titan Court, 3 Bishop Square, Hatfield, AL10 9NA, United Kingdom.

Most suitably the antibodies used may be as in the commercially available Bio-Rad Human cytokine Th-1/Th-2 27-plex kit (catalogue number #M500KCAF0Y from Bio-Rad Laboratories Ltd., Bio-Rad House. Maxted Road, Hemel Hempstead, Hertfordshire, HP2 7DX, United Kingdom). Most suitably detection of the cytokines of the signature of the invention are detected/assayed using this kit.

It is important to note that the prevailing view in the art is that stimulation of cells is required for use for analysis. The stimulation may be by presentation with bacteria, or may be by presentation with antigen or any other appropriate form of stimulation.

However, it should be noted that these types of stimulations are all directed at analysing recall responses. It is an advantage of the invention that unstimulated samples are analysed. In particular, it is an advantage that the sample is from unstimulated blood.

In particular when studying the key panel of 8 biomarkers, suitably the invention omits a stimulation step; suitably the invention does not comprise a stimulation step; suitably the invention excludes a stimulation step.

In some embodiments a ninth or further marker may be employed—a stimulation step may be employed for such further marker(s) if appropriate.

Most suitably the invention omits a stimulation step. Most suitably the invention does not comprise a stimulation step. Most suitably the invention excludes a stimulation step.

Nucleic Acid Detection

For example, the biomarkers may be detected in nucleic acid form, for example by detection of one or more mRNAs encoding the biomarkers.

If the skilled worker desires to read-out/detect nucleic acids via a microarray approach, reference is made to Anderson et al 2014 (N Engl J Med. 2014 May 1; 370(18):1712-23 ‘Diagnosis of childhood tuberculosis and host RNA expression in Africa.’ ILULU Consortium; KIDS TB Study Group.) mRNA technologies are suitably deployed in a laboratory setting.

In outline, mRNA detection may comprise the following steps:
stabilisation of RNA—can be done with a specific reagent,
extracted of RNA,
transcription to cDNA,
amplification,
array,
data read out.

Of course the person skilled in the art will realise that some steps are optional or may be combined, for example stabilisation/extraction may not be required if transcription can be performed directly on the sample. For example, array may not be required if the amplified material is assayed directly.

Thus in essence the required steps are:
extraction of nucleic acid
assay of nucleic acid to determine mRNA expression level of markers of interest
data read out.

For multiplex detection, suitably a fluorogenic oligonudeotide probe that is specific to the target gene/amplified target is used. Taqman probes are commonly used for multiplexing, but can also be used if multiplexing not required. Protocols areas stated by the manufacturer e.g. Applied Biosystems (5791 Van Allen Way, Carlsbad, Calif. 92008, US).

Different dyes can be used for the fluorogenic probes; examples that may be useful depending on the buffer conditions and type of thermal cycler are shown in the table below (Table from Qiagen Inc):

| | Excitation | Emission maximum |
|---|---|---|
| Dye | maximum (nm) | (nm)* |
| Fluorescein | 490 | 513 |
| Oregon Green | 492 | 517 |
| FAM | 494 | 518 |
| SYBR Green I | 494 | 521 |
| TET | 521 | 538 |
| JOE | 520 | 548 |
| VIC | 538 | 552 |
| Yakima Yellow | 526 | 552 |
| HEX | 535 | 553 |
| Cy3 | 552 | 570 |
| Bodipy TMR | 544 | 574 |
| NED | 546 | 575 |
| TAMRA | 560 | 582 |
| Cy3.5 | 588 | 604 |
| ROX | 587 | 607 |
| Texas Red | 596 | 615 |
| LightCycler Red 640 (LC640) | 625 | 640 |
| Bodipy 630/650 | 625 | 640 |
| Alexa Fluor 647 | 650 | 666 |
| Cy5 | 643 | 667 |
| Alexa Fluor 660 | 663 | 690 |
| Cy 5.5 | 683 | 707 |

*Emission spectra may vary depending on the buffer conditions.

Protocols are well known in the art, for example using the StepOne and/or StepOne Plus Real Time PCR Systems according to manufacturer's instructions (Applied Biosystems (5791 Van Allen Way, Carlsbad, Calif. 92008, US).

Other assays may be used if multiplexing was not desired, for example after reverse transcription, using dyes that bind double-stranded DNA and become florescent. For example SYBR green 1 (Qiagen Inc./Qiagen Ltd. Skelton House, Lloyd Street North, Manchester M 15 6SH, UK) may be used.

Primer probe assays useful in the invention can be designed, or purchased pre-designed, to the gene target of interest i.e. the biomarkers of the invention. For example, Applied Biosystems/ThermoFisher Scientific's (5791 Van Allen Way, Carlsbad, Calif. 92008, US) protocol for SYBR green 1 custom design (“Design and optimization of SYBR Green assays”) may be used, including the publicly available primer design tools discussed therein. This document is hereby incorporated herein by reference specifically for the primer/probe design protocols and nucleic acid detection teachings.

In case any further guidance is required, reference is made to the examples section below.

Determination of Quantiles/Deciles

Suitably converting each biomarker concentration determined into a decile value comprises the steps of:

(ci) comparing the concentration of each biomarker determined to a reference frequency distribution of concentrations of said biomarker; and

(cii) reading out the decile value from the frequency distribution for the concentration of said biomarker.

A “frequency distribution” shows a summarized grouping of data divided into mutually exclusive classes and the number of occurrences in a class. So it is possible to prepare a frequency distribution even with small numbers of data points such as 30 (e.g. exam scores of 30 children in a class). The larger the number of subjects used (i.e. data points), the more representative the distribution will be of the true population i.e. the more normally distributed the frequency distribution will be. Biological variables e.g. weight, height, blood pressure, Haemoglobin concentration, electrolytes in blood, cytokine measurements etc. are usually skewed. Thus, to get a normally distributed curve for such biological variables using a normal histogram for example, very large numbers of data points are needed which may be problematic. Therefore, non-parametric methods such as Kernel, which is a data-smoothing density estimator, are used. This is especially helpful when we want to present the representation of distribution of such data with a reasonable good sample size e.g. of 100 or more.

Therefore, when ‘frequency distribution’ is mentioned herein, suitably this may comprise a non-parametric equivalent such as a non-parametric density estimator, such as a data-smoothing density estimator, most suitably a Kernel density estimator.

Considering FIGS. 2 to 9, the Y-axes are appropriately labelled ‘Density’ because they indicate the Kernel Density Estimate.

In more detail, the Kernel frequency distribution is a data-smoothing statistical approach to displaying frequency distribution. Unlike the ordinary ‘histogram’, it is a non-parametric method which makes it very appropriate for our type of data, which like most biological data, does not follow the normal Gaussian distribution. The basic histogram displays frequency in number or proportion and the problems with histograms include the fact that it is not smooth and depends on the width of the bins (i.e. the bars) and end point of the bins which can be arbitrarily chosen. However, Kernel density estimate removes the dependence on end point of the bins by assuming no gap in the interval of cytokine measurements within a specified range, giving a smooth density estimate. The Y-axis of FIGS. 2 to 9 can simply be summarised as: “the probability density function of each respective marker.” Suitably the reference frequency distribution (or Kernel Density Estimate) is generated by measuring the concentration of the biomarker in a number of subjects, for example a minimum of 100 subjects, and compiling those measurements into a frequency distribution (or Kernel Density Estimate).

Alternatively the frequency distributions (Kernel Density Estimates) presented in FIGS. 2 to 9 herein may be used.

Suitably step (c) converting each concentration determined in (b) into a decile value comprises the steps of:

(ci) comparing the concentration of each biomarker determined in (b) to the corresponding reference frequency distribution or Kernel Density Estimate of concentrations of said biomarker selected from FIGS. 2 to 9; and

(cii) reading out the decile value from the frequency distribution or Kernel Density Estimate for the concentration of said biomarker.

In one embodiment, decile/quantile cutoffs may be augmented or replaced by absolute cut-offs expressed as absolute concentrations of the biomarker(s) in the sample.

Exemplary absolute cut-off values are provided in the table below:

| | Specific | | Absolute |
|---|---|---|---|
| | quantile | Concentration | cut-off |
| | cut-off | range for specific | concentrations i.e. ≥ |
| Biomarker | value | quantile (pg/ml) | XX |
| IL-1ra | 3 | 76.0-99.5 | 76.0 |
| IL6 | 6 | 759.0-1203.0 | 759.0 |
| IL-7 | 8 | 167.0-269.0 | 167.0 |
| IL-8 | 9 | 20196.0-25900.0 | 20196.0 |
| IL-12p70 | 9 | 596.0-776.0 | 596.0 |
| FGF-basic | 3 | 125.5-145.0 | 125.5 |
| IP-10 | 4 | 3096.5-4537.0 | 3096.5 |
| VEGF | 9 | 3378.0-5381.0 | 3378.0 |

In one embodiment, decile/quantile cutoffs may be augmented or replaced by the concentration range for the specific quantile of interest expressed as the range of absolute concentrations of the biomarker(s) in the sample. Exemplary concentration ranges are provided in the table above.

### Point of Care Test

In many embodiments, the skilled operator may choose to analyse the concentrations of the markers in a laboratory or test facility.

The invention may also be applied as a point of care test. Suitably the invention may also be applied as a bedside test.

When the invention is a point of cared bedside test, suitably the sample is blood or plasma. When the invention is a point of care bedside test, suitably the markers are analysed in protein form.

When the invention is a point of care/bedside test, suitably the detection is immunological detection.

When the invention is a point of care/bedside test, suitably the test is the a format of a lateral flow assay.

### Predictive Values/Applications of the Method

The table below provides additional results of predictive values of the biosignature. We show and compare the PPV and NPV of the biosignature to prior art methods. We further show it has demonstrable comparable performance to the gold standard of culture. The high specificity and the positive predictive value of the invention lends itself to change treatment decisions. This enables accurate prescription for TB detected according to the invention. This also avoids waste of resources in prescription of unnecessary drugs. This further demonstrates the utility of the invention.

Diagnostic accuracy of tests relative to a
‘composite reference standard’

| | ‘All TB diagnosis’ as a | | | |
|---|---|---|---|---|
| | Composite reference standard | | | |
| | Sensitivity | | | |
| | (%) | Specificity (%) | PPV (%) | NPV (%) |
| | (95% CI) | (95% CI) | (95% CI) | (95% CI) |
| Chest X-ray | 76 | 34 | 34 | 76 |
| (prior art) | (62-86) | (26-43) | (25-43) | (62-87) |
| Clinical algorithm | 15 | 99 | 89 | 73 |
| (prior art) | (7-28) | (95-100) | (52-100) | (65-79) |
| Culture | 36 | 100 | 100 | 78 |
| (prior art) | (23-50) | (97-100) | (82-100) | (71-84) |
| Biosignature | 23 | 99 | 92 | 75 |
| (invention) | (12-36) | (95-100) | (64-100) | (67-81) |

Further Applications

The key set of 8 biomarkers are advantageously assessed from unstimulated samples. However, in some embodiments it may be helpful to further assess a ninth or further marker; suitably such a ninth or further marker comprises a stimulated marker such as EC-stimulated VEGF. Suitably the cutoff for EC-stimulated VEGF is decile 2. For the avoidance of doubt, details of the ‘VEGF’ biomarker of EC-stimulated VEGF areas above in the key panel of 8 biomarkers of the invention (‘VEGF’).

A method for aiding the diagnosis of TB in a subject, the method comprising;
(a) providing a sample from said subject, said sample being selected from the group consisting of: blood, serum and plasma;
(b) determining the concentration in said sample of the following biomarkers: IL-1ra, IL6, IL-7, IL-8. IL-12p70. FGF-basic. IP-10, and VEGF;
(c) converting each biomarker concentration determined in (b) into a decile value; and
(d) converting each decile value into a binary presence or absence by comparing the decile values of (c) to the following specific quantile cut-off values:

| Biomarker | Specific quantile cut-off value |
|---|---|
| IL-1ra | 3 |
| IL6 | 6 |
| IL-7 | 8 |
| IL-8 | 9 |
| IL-12p70 | 9 |
| FGF-basic | 3 |
| IP-10 | 4 |
| VEGF | 9 |

wherein a decile value matching or exceeding the specific quantile cut-off value is converted into the binary presence of the biomarker, and a decile value lower than the specific quantile cut-off value is converted into the binary absence of the biomarker;

wherein detecting the presence of each of said biomarkers indicates an increased likelihood that the subject has TB.

A method for differentiating TB from OD in a subject, the method comprising;

carrying out steps (a) to (d) above

wherein detecting the presence of each of said biomarkers indicates that the subject has TB.

A method of collecting information useful in diagnosis of TB in a subject, the method comprising:

carrying out steps (a) to (d) above

wherein detecting the presence of each of said biomarkers identifies the subject as having TB.

A method for the diagnosis of TB in a subject, the method comprising;

carrying out steps (a) to (d) above

wherein detecting the presence of each of said biomarkers provides the diagnosis that the subject has TB.

A method for selecting a subject to receive treatment for TB, the method comprising;

carrying out steps (a) to (d) above

wherein detecting the presence of each of said biomarkers selects said subject to receive said treatment.

A method comprising the steps of selecting a subject to receive treatment for TB by; carrying out steps (a) to (d) above

wherein detecting the presence of each of said biomarkers selects said subject to receive said treatment; and administering said treatment to said subject.

A method of treating TB in a subject comprising administering a regimen of 2HRZE/4HR (2 months HRZE followed by 4 months HR wherein H=isoniazid, R=rifampicin, Z=pyrazinamide, E=ethambutol) to a subject determined to have each of the following biomarkers: IL-1ra, IL6, IL-7, IL-8, IL-12p70, FGF-basic, IP-10, and VEGF. The invention also relates to said method further comprising testing the subject prior to the administering step to determine that the subject has the following biomarkers: IL-1ra, IL6, IL-7, IL-8, IL-12p70, FGF-basic, IP-10, and VEGF. Suitably testing is carried out by carrying out steps (a) to (d) above.

In so far as the embodiments of the invention described above are implemented, at least in part, using software-controlled data processing apparatus, it will be appreciated that a computer program providing such software control, and a storage medium by which such a computer program is stored, are envisaged as aspects of the present invention. Clearly in several of the methods or processes of the invention, one step (typically step (a)) comprises providing a sample from the subject—dearly that step would not typically be performed using software-controlled data processing apparatus; suitably that step is manually executed, or omitted, in embodiments implemented using software-controlled data processing apparatus.

Thus the invention relates to an apparatus such as a computer comprising logic, circuitry or code configured to carry out the method as described above.

Thus the invention relates to a computer program product operable, when executed on a computer, to perform the method as described above.

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

### BRIEF DESCRIPTION OF THE DRAWINGS

Embodiments of the present invention will now be described by way of example, with reference to the accompanying drawings, in which:

FIG. 1 shows optimal LASSO models with cytokine covariates, adjusted for age and origin. Panels a, b, c: Optimal LASSO model, adjusted for age and origin, determined by 5-fold cross validation in training set. Panels d, e, f: box-and-whisker plot showing probability of TB disease in the bacteriologically-confirmed TB, clinically diagnosed TB and OD subjects in the training set as predicted by the identified biosignature. Panels g, h, i: AUC showing discriminating ability of identified biosignature to classify confirmed TB from OD in the independent test set

FIG. 2 shows a frequency distribution for IL-1ra

FIG. 3 shows a frequency distribution for IL-6

FIG. 4 shows a frequency distribution for IL-7

FIG. 5 shows a frequency distribution for IL-8

FIG. 6 shows a frequency distribution for IL-12p70

FIG. 7 shows a frequency distribution for FGF-basic

FIG. 8 shows a frequency distribution for IP-10

FIG. 9 shows a frequency distribution for VEGF

FIG. 10 shows a lateral flow device.

FIG. 11 shows the multiplex detection format of a lateral flow device.

### EXAMPLES

### Methods

### Brief Cohort Description

Children aged less than 15 years who were exposed to an adult infectious TB case in the household setting were actively traced and screened for symptoms suggestive of TB disease in the respective households. Those with suspected intrathoracic TB disease thereafter had further detailed clinical evaluation and investigations to ascertain their TB disease status. A total of 173 child TB contacts with suspected intrathoracic TB disease, prospectively recruited both in The Gambia (n=150) and United Kingdom (n=23), were included in the biosignature discovery experiments using an immuno-epidemiological approach.

### Whole Blood Stimulation Assay (WBA)

For the Gambia cohort, a WBA was set up at the recruitment within four hours of venepuncture. 100 μl of undiluted heparinised whole blood was incubated in duplicates with M.tb antigens ESAT-6/CFP-10 fusion protein (EC; 10 μg/ml final concentration; kindly provided by Professor Tom Ottenhoff, Leiden University Medical Center, The Netherlands) and positive (PHA-L, Sigma Chemicals, UK; 10 μg/ml final concentration) and negative (RPMI 1640 medium; BioWittaker, Verviers, Belgium) controls. After overnight incubation at 37° C. with 5% CO., supernatants were harvested, duplicates pooled and stored at −20° C. prior to analysis. Samples were added to this cohort from an equivalent set up of a household contact study in the UK, conducted by BK. For the children from the UK cohort, we had also obtained the relevant demographic and clinical data and derived supernatants from IGRA (Quantiferon-TB Gold In-Tube (QFT-G) test (Cellestis, Australia). Similar to our in-house assay, this commercially available in-vitro IFN-γ release assay uses stimulation of fresh whole blood in three separate tubes containing M.tb antigens (ESAT-6, CFPIO and TB 7.7), positive (Phytoheamaglutinin-L) and negative (Nil) controls respectively. These samples were shipped frozen to The Gambia for joint analysis.

The WBA supernatants from the Gambian children and the QFT supernatants from the children from the UK cohort were used for a multiplex cytokine detection assay (MCA). The MCA was carried out on site in the MRC TB Immunology laboratory in The Gambia, with the Gambian and UK samples randomly distributed over the multiplex plates.

### Multiplex Cytokine Detection Assay (MCA)

We carried out a comprehensive MCA by Luminex using the unstimulated and EC-stimulated WBA supernatants of the Gambian children and QFT supernatants (from antigen and nil QFT tubes) of the children from the UK cohort. Culture supernatants were analysed using the Bio-Rad Human cytokine Th-1/Th-2 27-plex kit according to the manufacturer's instruction and as described previously [1]. Cytokines assessed were: IL-1b, IL-1ra, IL-2, IL-4, IL-5, IL-6, IL-7, IL-8, IL-9, IL-10, IL-12p70, IL-13, IL-15, IL-17, Eotaxin, FGF basic, G-CSF, GM-CSF, IFNγ, IL-10, MCP-1(MCAF), MIP-1a, MIP-1b, PDGF-bb, RANTES, TNFα and VEGF. Following pre-wetting of the filter plate, 50 μl of bead suspension was added to each well and washed twice. 50 μl of samples and standards, tested singly and in duplicates respectively, were then added and the plate sealed and shaken for 30 seconds at 1100 rpm and then incubated for one hour at 300 rpm. The plate was washed three times, 25 μl of pre-diluted detection antibody was added and the plate shaken and incubated for 30 minutes at 300 rpm in the dark. After washing, 50 μl of 1× streptavidin-PE was added to each well and incubated for 10 minutes with shaking at 300 rpm. The plate was again washed and resuspended in 125 μl of the assay buffer, sealed, mixed and immediately read on the Bio-plex analyser using Bioplex manager software (version 4.0; Bio-Rad, USA) and a low photomultiplier tube (PMT) setting. Cytokine concentrations below the level of detection—reported as ‘OOR’ in the Bioplex software—were calculated as zero in the analysis.

### Statistical Analysis

We analysed the data obtained from the multiplex cytokine assay (MCA) of unstimulated and EC-stimulated whole blood culture supernatants for the identification of the host-specific multicytokine biosignature associated with TB in children. For this analyses, the unstimulated and antigen-specific cytokine responses from the 27-plex MCA were analysed as separate variables. We randomly assigned the study subjects into a training set (80% of subjects) and an independent test set (20%). We then used Generalized Linear Model (GLM) applying Least Absolute Shrinkage and Selection Operator (LASSO) penalty to fit logistic regression models in the training set adjusting for age in years and origin of sample, initially with bacteriologically confirmed TB (gold standard) compared to other respiratory diseases mimicking TB but not TB (OD group) as the binary outcome variable. The LASSO model applies a maximum penalised likelihood to the absolute size of the regression coefficients, shrinking them towards zero i.e. an L1 norm penalty is applied to the regression coefficients. This procedure results in both variable selection (some regression coefficients equal zero) and estimates of non-zero regression coefficients shrunk towards zero. This methodology is suitable to our cytokine data in which there were very many measures, many of which could potentially be highly correlated.

We fitted the cytokine covariates as categorical and continuous variables and as a combination of both. Categorical cytokine covariates were constructed by a split of the cytokine values into deciles by dividing the frequency distribution of each cytokine value into 10 equal-sized quantiles, which were then fitted into the model as 10 binary variables for each cytokine using each of 10 quantiles as a cut off. The optimal LASSO model was determined using a 5-fold cross-validation in the training set, which was subsequently applied to classify bacteriologically confirmed TB from OD in the independent test set naïve of origin. The optimal model was defined as the model with the highest penalty parameter (‘lambda) resulting in the smallest prediction error and the best mean cross-validation AUC. The cross-validation process accounts for, and replaces the classical method of adjusting for multiple testing. In addition, it naturally protects against over-fitting and it is a way of assessing how a model will generalise to an independent dataset. The prediction performance of the optimal LASSO model was evaluated by estimating prediction probabilities for TB and area under the receiver operating characteristics curves (AUC).

### Results

Overall, 53 children from the combined cohorts were diagnosed with TB and started on standard TB treatment; 24 had bacteriologically-confirmed TB and 29 had TB diagnosed based on clinical and radiological features with no positive microbiological tests. One hundred and twenty were diagnosed and treated for OD. In detail, thirty-five of the 150 Gambian children had TB, comprising 16 bacteriologically-confirmed and 19 clinical diagnosed TB cases, while 115 had OD. Of the 23 children from the UK, 18 were diagnosed with TB (8 confirmed and 10 clinically diagnosed TB) while 5 had OD. None of the children recruited from The Gambia or UK was HIV infected. Table 1 shows that the distribution of the baseline profiles of the children from Gambia and UK were comparable.

TABLE 1
Demographic profile of children by origin

| | Total | | The Gambia | | United Kingdom | |
|---|---|---|---|---|---|---|
| Characteristics | N = 173 | | N = 150 | | N = 23 | |
| Age: | | | | | | |
| Years, Median | 6 | (3-9) | 6 | (3-9) | 7 | (3-12) |
| (IQR) | | | | | | |
| <5 years, n/N (%) | 65/173 | (38) | 55/150 | (37) | 10/23 | (43) |
| Gender: | | | | | | |
| Male, n/N (%) | 88 | (51) | 76 | (51) | 12 | (52) |
| HIV test: | | | | | | |
| Positive, n/N (%) | 0 | | 0 | | 0 | |
| BCG Scar: | | | | | | |
| Present, n/N (%) | 117/164 | (71) | 99/141 | (70) | 18/23 | (78) |
| TST: | | | | | | |
| ≥10 mm, n/N (%) | 98/171 | (57) | 82/149 | (55) | 16/22 | (72) |
| IGRA: | | | | | | |
| Positive, n/N (%) | 120/173 | (69) | 103/150 | (69) | 17/23 | (74) |

### Pattern of Cytokine and Chemokine Production in Confirmed TB vs OD

The concentrations of cytokines and chemokines obtained by a 27-plex multiplex cytokine analysis of unstimulated supernatants and EC-stimulated supernatants from children with bacteriologically confirmed TB were compared with the levels in children with OD by multivariable linear regression analyses, adjusting for age in years and origin. The unstimulated and EC-specific values (i.e. EC-stimulated minus unstimulated negative control values) for each cytokine or chemokine were analysed as separate variables.

TABLE 2
Mean difference in concentration (pg/ml) of the 27-markers: Confirmed TB vs OD

| | Unstimulated values (pg/ml) | | | ESAT-6/CFP-10-specific values (pg/ml) | | |
|---|---|---|---|---|---|---|
| | Confirmed TB vs OD | | | Confirmed TB vs OD | | |
| | Mean | | | Mean | | |
| Analytes | difference | 95% CI | P-value^{Ω} | difference | 95% CI | P-value^{Ω} |
| IL-1b | 307.7 | −961.8-1577.1 | 0.633 | 41.3 | −4025.6-4108.3 | 0.984 |
| IL-1ra | 118.2 | 13.4-222.9 | 0.027 | 336.9 | −359.5-1033.4 | 0.340 |
| IL-2 | 43.9 | −71.7-159.6 | 0.454 | 4995.1 | 1981.2-8009.0 | 0.001 |
| IL-4 | 58.8 | −2.3-119.9 | 0.059 | 76.4 | −52.4-205.2 | 0.243 |
| IL-5 | 36.8 | −17.8-91.4 | 0.185 | −39.9 | −670.3-590.3 | 0.900 |
| IL-6 | 1289.8 | −1193.7-3773.3 | 0.306 | −555.1 | −4207.1-3097.0 | 0.764 |
| IL-7 | 181.8 | 90.0-273.6 | 0.000 | −82.1 | −140.9 to −23.3 | 0.007 |
| IL-8 | 2555.1 | −388.3-5498.6 | 0.088 | −1800.4 | −5554.9-1954.1 | 0.345 |
| IL-9 | 81.6 | −28.1-191.1 | 0.144 | −61.9 | −281.8-157.9 | 0.578 |
| IL-10 | 51.2 | −140.7-243.2 | 0.598 | 28.1 | −3357.7-3413.9 | 0.987 |
| IL-12p70 | 153.5 | 43.0-263.9 | 0.007 | 7.1 | −158.9-173.0 | 0.933 |
| IL-13 | 129.1 | 26.7-231.6 | 0.014 | 728.2 | −331.4-1787.8 | 0.176 |
| IL-15 | 1258.5 | 468.4-2048.6 | 0.002 | −237.7 | −928.7-453.3 | 0.498 |
| IL-17 | 116.3 | 11.9-220.7 | 0.029 | 122.6 | −20.3-265.5 | 0.092 |
| Eotaxin | 214.2 | 75.5-352.9 | 0.003 | 13.4 | −138.5-165.2 | 0.862 |
| FGF- | 114.9 | 13.6-216.1 | 0.026 | 65.5 | −59.7-190.8 | 0.303 |
| GCSF | 222.9 | 51.3-394.6 | 0.011 | 1009.0 | −793.6-2811.6 | 0.270 |
| GMCSF | 41.4 | −185.6-268.4 | 0.719 | 236.1 | −81.0-553.2 | 0.143 |
| IFN-γ | 81.4 | 4.8-158.0 | 0.038 | 2148.9 | −1144.4-5442.4 | 0.199 |
| IP-10 | 4631.9 | 877.2-8386.7 | 0.016 | −434.3 | −4602.8-3734.2 | 0.837 |
| MCP1- | 4739.8 | −314.5-9794.1 | 0.066 | −3830.4 | −9061.8-1400.9 | 0.150 |
| MIP1a | 2721.4 | −337.5-5780.3 | 0.081 | −939.9 | −4887.5-3007.8 | 0.639 |
| MIP1b | 4400.8 | 364.0-8437.5 | 0.033 | −3997.5 | −8244.7-249.8 | 0.065 |
| PDGF | 326.1 | −1053-1705.3 | 0.641 | 437.8 | −575.8-1451.2 | 0.395 |
| RANTES | −106.3 | −1446.0-1233.4 | 0.876 | 273.5 | −943.5-1490.6 | 0.657 |
| TNF-α | 273.8 | −1.7-549.3 | 0.051 | −399.0 | −4033.0-3234.9 | 0.828 |
| VEGF | 1977.7 | 1116.1-2839.2 | 0.000 | −491.6 | −1224.0-240.8 | 0.187 |

^{Ω}Adjusted for age in years and origin
95% CI = 95% confidence interval of mean difference

As shown in Table 2, of the 27 cytokines and chemokines analysed, the unstimulated concentrations of IL1ra, IL7, IL12p70, IL3, IL15, IL17, Eotaxin, basic FGF, GCSF, IFN-γ, IP10, MIP1b and VEGF were significantly higher in children with confirmed TB compared to children with OD. Of the EC-specific concentrations of all the analytes, only IL2 and IL7 were significantly different between the two groups. Furthermore, we found that the concentrations in unstimulated samples were significantly higher for all the analytes in children from the UK compared to Gambian children regardless of age or diagnosis (p-value <0.001 for all), with the exception of unstimulated-IL7 value in which there was no significant difference (data not shown). On the contrary, the EC-specific concentrations of the analytes were significantly lower in children from the UK compared to Gambian children regardless of age and diagnosis (p-value <0.001 in all), with the exception of EC-specific concentrations of IL2, IL5, IL7, 113, IFN-γ and RANTES in which there were no significant differences.

### Identification of a Host Specific Biosignature for the Diagnosis of Childhood TB

Using GLM with LASSO penalty to fit a binary logistic regression model, adjusting for age in years and origin and with 5-fold cross-validation in the training set, a combination of nine categorical cytokines optimally predicted TB or OD with a mean cross-validation AUC of 0.82. Each of the nine cytokines is a binary variable with a cut-off value.

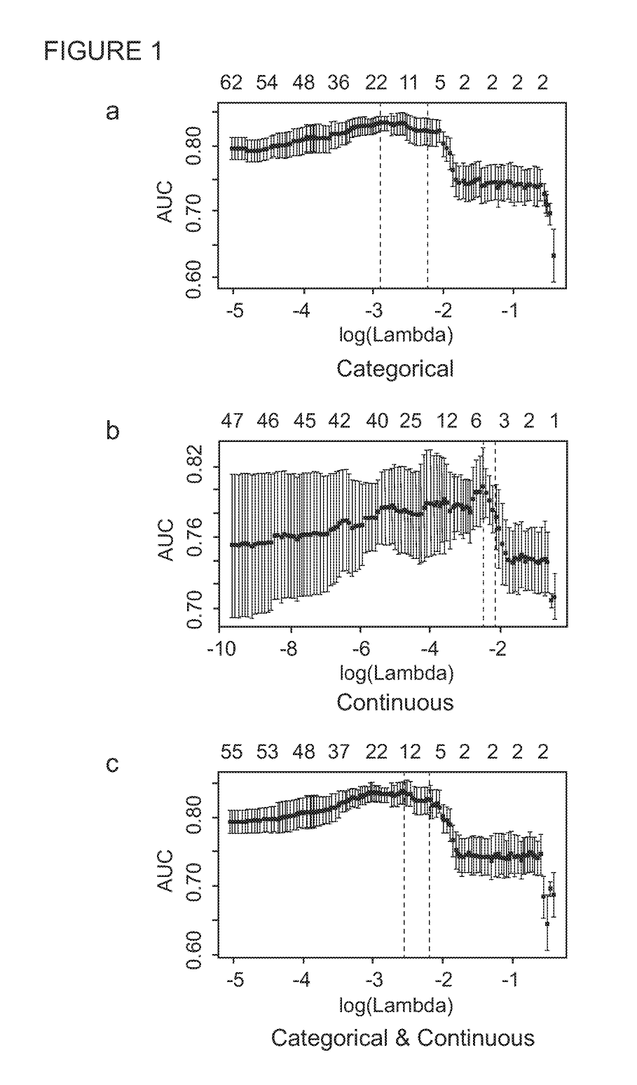

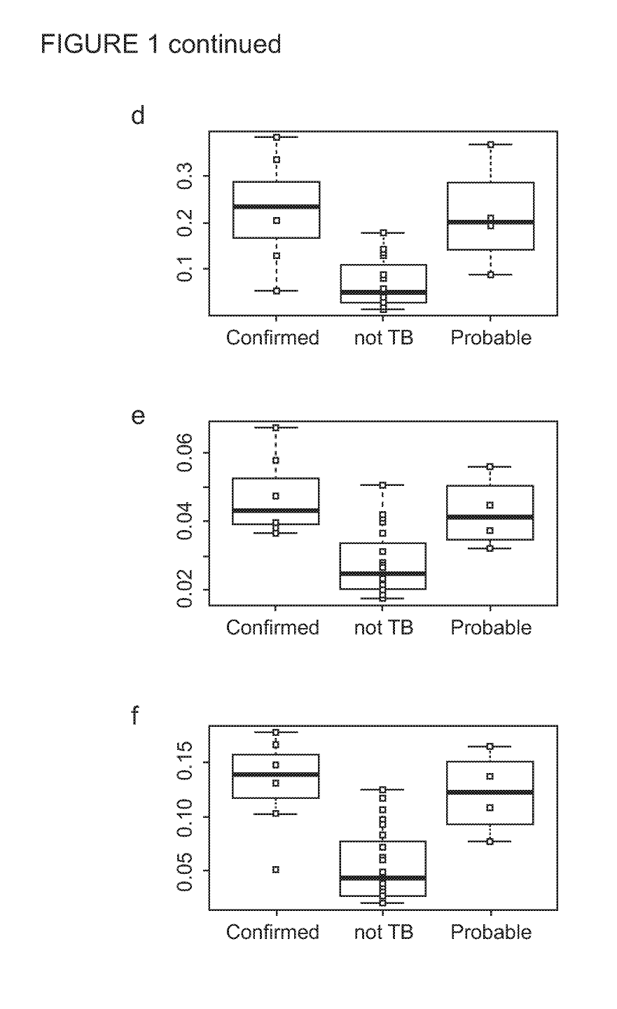

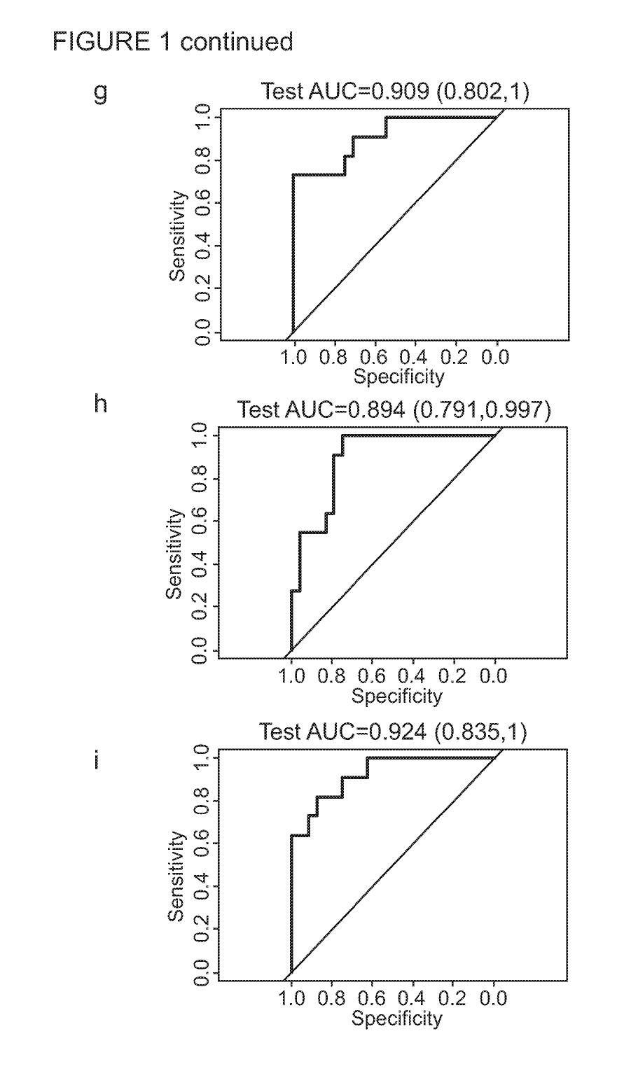

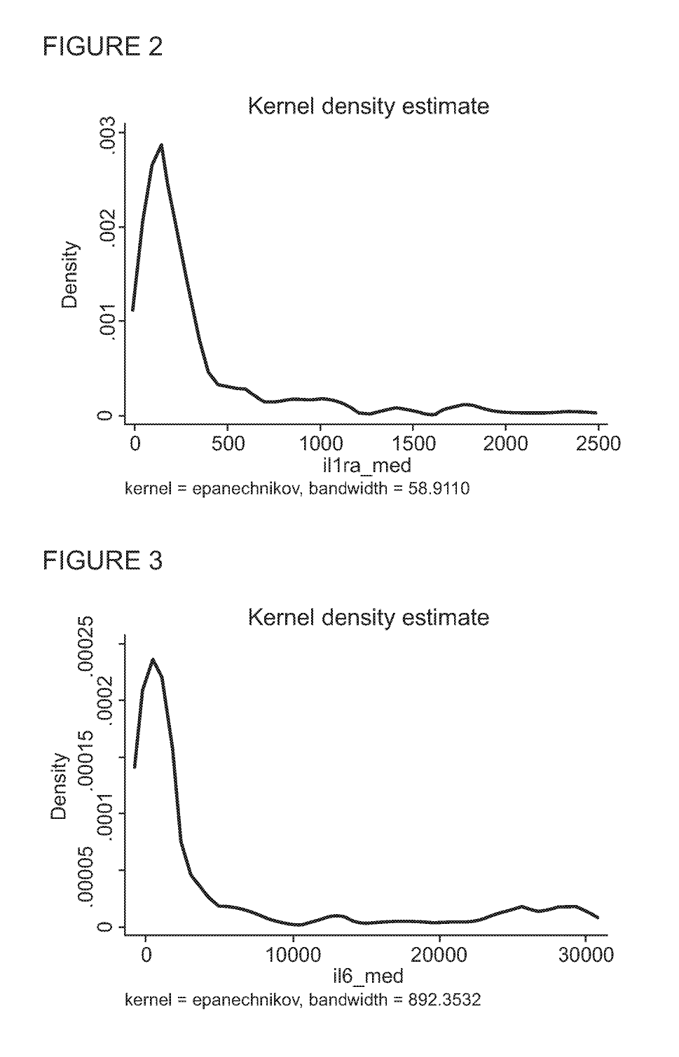

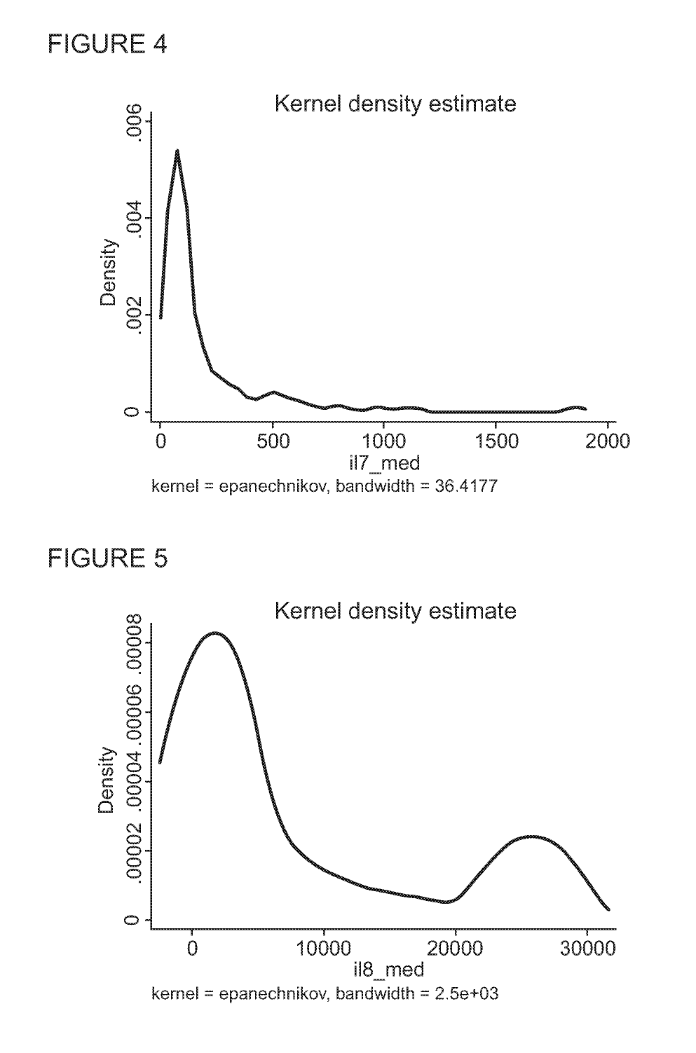

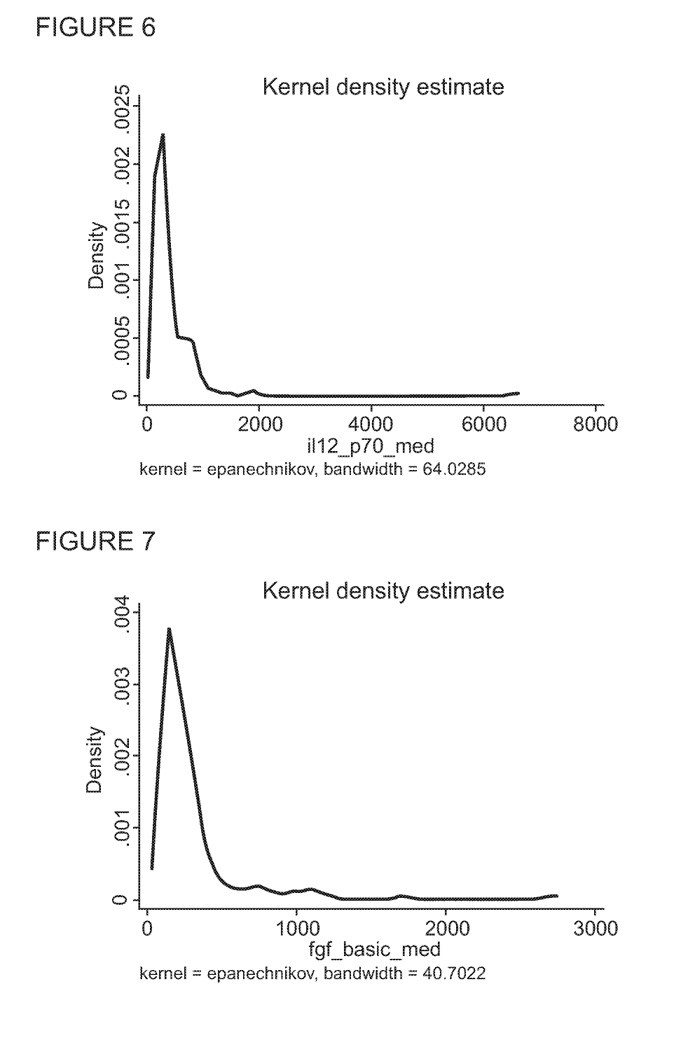

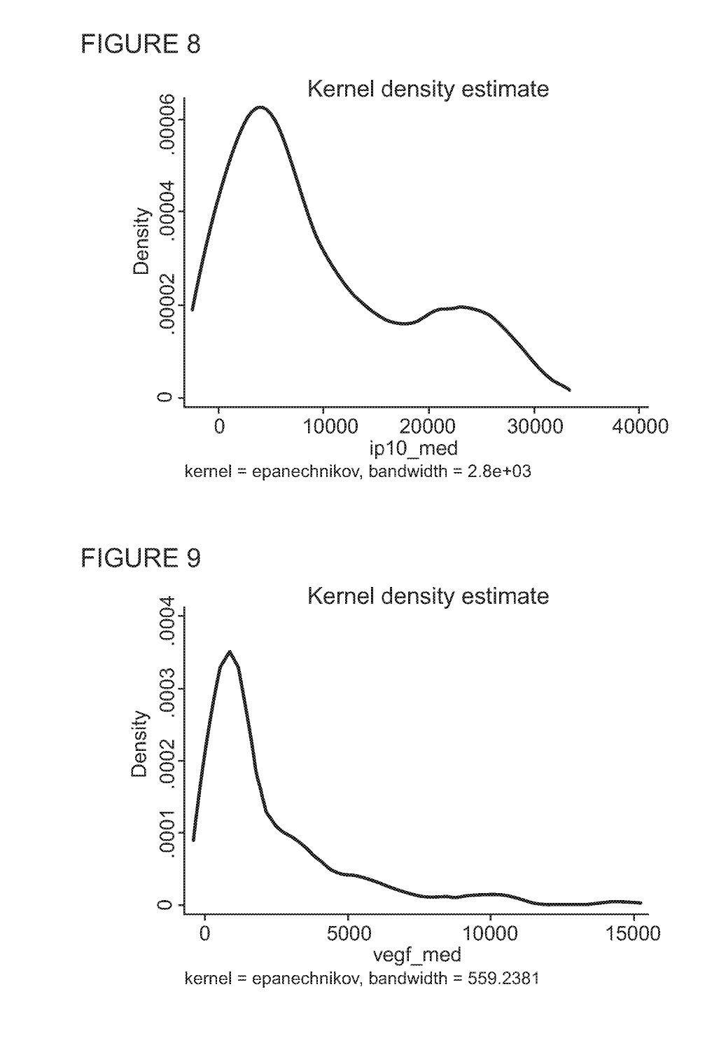

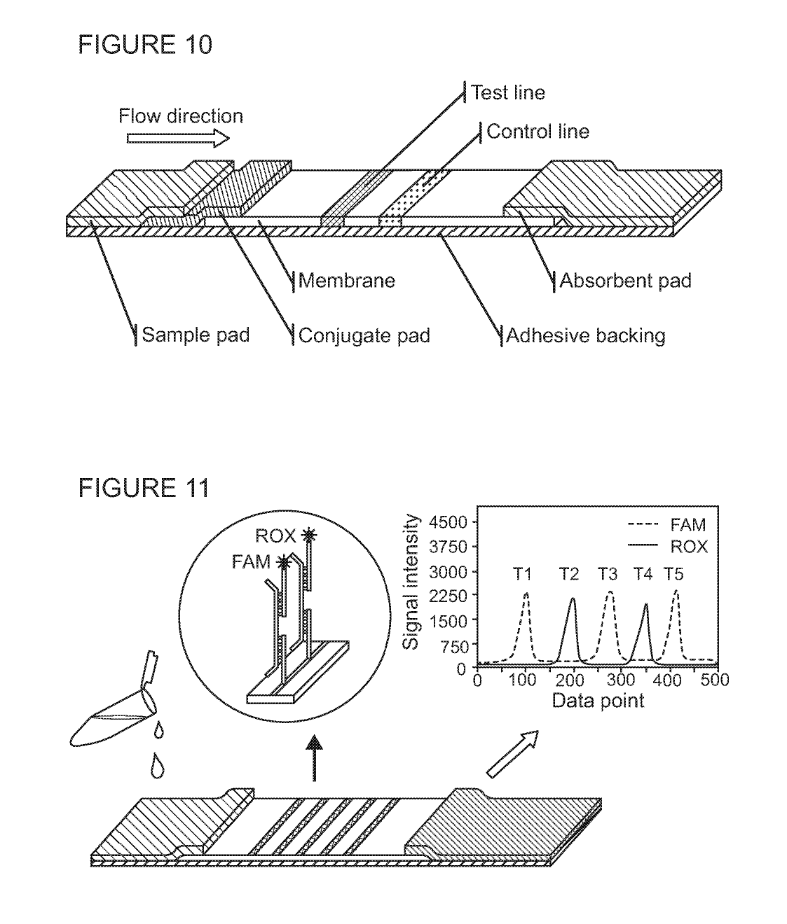

The cytokines—with the specific quantile cut-off value for each in bracket—were: unstimulated IL-1ra (3), IL6 (6), IL-7 (8), IL-8 (9), IL-12p70 (9), FGF-basic (3), IP-10 (4), VEGF (9) and EC-stimulated VEGF (2). When applied to the independent test set, this model reliably classified confirmed TB from OD with an AUC of 0.91 (95% CI 0.80-1.0) as depicted graphically in FIG. 1.

### Diagnostic Accuracy and Added Value of the Multicytokine Biosignature

The quantile specific cut-off values for each cytokine in the biosignature enabled us to convert the biosignature into a binary—positive or negative—test. We investigated the diagnostic accuracy of the biosignature as two separate variables i.e. “biosignature 1” (combination of all the 9 identified cytokines) and “biosignature 2” (combination of only the 8 cytokines from unstimulated supernatants). When we compared the results of the novel multicytokine biosignatures to the disease certainty classification (i.e. bacteriologically-confirmed TB (=confirmed TB), clinically diagnosed TB (=probable TB) and other respiratory diseases but not-TB (OD) as defined by the WHO [2], ‘biosignature1’ was positive in 8% of confirmed TB and 7% of clinically diagnosed TB cases. However ‘biosignature2’ had a relatively higher sensitivity with positive results in 21% and 24% of confirmed and clinically diagnosed TB cases respectively. Both biosignature versions were negative in 119 of 120 OD cases giving a very high specificity of 99.2% (Table 3).

TABLE 3
Performances of biosignatures relative to TB disease certainty
classification
WHO Revised TB Case Definition*

| | | Clinically | |
|---|---|---|---|
| | Confirmed TB | diagnosed | OD (not-TB) |
| | (N = 24), n (%) | TB | (N = 120), n (%) |
| ‘Biosignature1’ + | 2 | 2 | 1 (0.8) |
| ‘Biosignature1’ − | 22 | 27 | 119 (99.2) |
| ‘Biosignature2’ + | 5 | 7 | 1 (0.8) |
| ‘Biosignature2’ − | 19 | 22 | 119 (99.2) |

+ = test positive;
− = test negative;
% = column percentage i.e. n/N
(*W.H.O. Definitions and reporting framework for Tuberculosis - 2013 revision, Geneva, Switzerland. www.who.int/iris/bitstream/10665/79199/1/9789241505345_eng.pdf [Accessed 3 Dec. 2014])

Using a composite reference standard of all children diagnosed with active TB disease, ‘biosignature2’ derived from only markers in unstimulated supernatants was positive in 12 of all the 53 children diagnosed with active TB disease giving a sensitivity of 23% (95% CI 12-36). As individual tests, the sensitivity of ‘biosignature2’ was significantly higher than that of smear microscopy (5.7%; p-value=0.020) but comparable to that of M.tb culture (35.9%; p-value=0.127). The combination of ‘biosignature2’ and smear microscopy were positive in 15 of 53 children with active TB disease giving a sensitivity of 28.3%, which was significantly higher than the sensitivity of smear microscopy alone (5.7%; p<0.001). Similarly, ‘biosignature2’ combined with M.tb culture had a sensitivity of 49.1%, which was significantly higher than the sensitivity of M.tb culture used alone (35.9/c; p<0.001). The sensitivity of ‘biosignature2’ combined with M.tb culture was significantly higher than that of ‘biosignature2’ combined with microscopy (p<0.001), but comparable to the sensitivity of the combination of ‘biosignature2’, microscopy and M.tb culture (p=0.320). The use of ‘biosignature2’ in combination with these diagnostic tests did not result in any change in the specificity of the tests.

### SUMMARY

Since host immune factors such as IFN-γ have been shown to be important, but insufficient to confirm or exclude TB [3, 4], the aim of this study was to investigate cytokines other than IFN-γ that may help to differentiate TB from OD in Gambian and UK children, since the distinction of these two clinical presentations is important to initiate the right therapy. Previous studies have reported other cytokines such as TNF-α, IL-12(p40), IL-6, IL-10, IL-18 and IL-17, FGF and VEGF that have been found to be important in the immune response against M.tb and/or in distinguishing TB from OD [1, 5, 6].

We identified a unique, quantile-specific, 9-cytokine biosignature that optimally distinguished bacteriologically-confirmed TB from OD irrespective of age and origin of the children. The biosignature also predicted the probability of active TB disease in children with clinically-diagnosed TB that was comparable to that of bacteriologically-confirmed TB cases. Specifically, we used an age and origin adjusted LASSO regression model to identify a quantile-specific combination of unstimulated IL-1ra, IL-6, IL-7, IL-8, IL-12p70, basic FGF, IP-10, VEGF and EC-stimulated VEGF that optimally distinguished between bacteriologically-confirmed TB and OD with an AUC of 0.91 in an independent test set. The performance of this biosignature was regardless of sensitization to M.tb in the clinical outcome groups, while eight of the 9-cytokines in the biosignature were from unstimulated supernatants. A major strength of our study is the prospective approach used in an exclusively paediatric active case finding study setting while the identified biosignature in our cohort contains cytokines known to be closely associated with TB immunity.

We investigated separately the diagnostic accuracy of the 8-cytokines biosignature comprising only the markers from unstimulated supernatants, and our full 9-cytokine biosignature by comparing their results to disease certainty classifications according to WHO case definitions and a composite reference standard of all children diagnosed with active TB disease. We found that while the unstimulated 8-cytokine biosignature had a relatively higher sensitivity than the full 9-cytokine biosignature, both biosignature versions distinguished active TB disease from OD with a very high specificity of 99.2%. The unstimulated 8-cytokine biosignature detected a comparable number of TB cases among all children diagnosed with active TB disease in our study relative to M.tb culture, and demonstrated substantial added value when combined with routine TB diagnostic tests. It showed a comparably higher specificity but a lower sensitivity relative to a risk score based on a 51-whole blood gene transcript that was identified in a multi-country childhood TB biomarker study in south and eastern Africa, as well as a three marker combination of TNF-α, IL-12(p40) and IL-17 in antigen stimulated whole blood supernatants of adult Gambians [1, 7]. However, the specificity of this paediatric biosignature is comparable to that of the combination of IL-13, FGF and IFN-γ in ex vivo sputum samples in another study in adults in The Gambia, which resulted in 96% correct classification of consecutively recruited culture-confirmed TB cases from OD with a sensitivity of 85% and specificity of 96% [6].

A number of factors makes this unstimulated multicytokine biosignature a particularly promising approach for use in high TB burden countries. First, the quantile-specific cut-off values for each of the component cytokines mean the readouts can be easily converted into a binary test with either a positive or negative result, which makes it more easily interpretable. Secondly, this multicytokine biosignature, derived from only markers in unstimulated supernatants, had similar epidemiological properties to M.tb culture but is potentially not subject to the same time delay or risk of contamination as culture. Thirdly, it has a demonstrable potential to reduce presumptive treatment of TB disease in children in primary care settings in developing countries where TB diagnosis is mostly based on the use of smear microscopy, as well as at referral centres with X-ray, Xpert and/or culture facilities. This is because of the substantial increases in the number of children who would be deemed to have active TB disease when used in combination with the routine diagnostic tests. Fourthly, this unstimulated multicytokine biosignature could potentially be measured directly in serum or plasma samples without the additional cost, training or infrastructure needed for antigen stimulation and incubation in the laboratory. Thus the 8-cytokine biosignature is the most preferred embodiment of the invention.

### Example 2: Nucleic Acid Based Detection

In this example we describe processing of samples to illustrate the application of the invention/gene signature in aiding diagnosis of TB (such as childhood TB) via nucleic acid detection, such as RNA (mRNA) detection.

1. Sample Collection:

The blood sample is collected into a tube containing a stabilising agent for RNA, such as a PaxGene tube or tempus tube.

Alternatively trizol is added to sample.

Such sample may be stored in fridge or freezer until processing.

If stored in the fridge, the storage time is days, if stored in the freezer the storage time can be months.

2. Sample Processing

Depending on the collection tube and stabilising agent, suitable commercially available RNA extraction kit(s) are selected and used according to the manufacturer's instructions.

In this example, Qiagen kits containing spin columns and wash buffers for RNA extraction are used.

Total RNA including micro RNA is extracted using these established methods.

3. Reverse Transcription

In order to obtain DNA which can be amplified, a transcription reaction needs to first convert mRNA to cDNA. This is done by addition of RT random primers, dNTPs, Reverse Transcriptase and RT buffers in the appropriate amounts, followed by thermal cycling incubation as is known in the art.

In this way, the RNA is reverse transcribed into DNA.

4. Amplification

cDNA can then be amplified using primers and/or probes specific for the transcripts of interest of the biomarkers as described above, suitably together with primers and/or probes specific for reference genes for normalisation.

For convenience, in this example primers and/or probes for each cytokine in question, internal controls and endogenous control genes are added to a mastermix along with the cDNA template, and triplicate PCR reactions in either single-plex or multi-plex are carried out using a real-time PCR instrument.

### REFERENCES

1. Sutherland J S, de Jong B C, Jeffries D J, Adetifa I M, Ota M O. Production of TNF-alpha, IL-12(p40) and IL-17 can discriminate between active TB disease and latent infection in a West African cohort. PloS one 2010: 5(8): e12365.
2. W.H.O. Definitions and reporting framework for Tuberculosis—2013 revision., Geneva, Switzerland. www.who.int/iris/bitstream/10665/79199/1/9789241505345_eng.pdf [Accessed 3 Dec. 2014], 2013.
3. Kaufmann S H. Fact and fiction in tuberculosis vaccine research: 10 years later. The Lancet infectious diseases 2011: 11(8): 633-640.
4. Flynn J L, Chan J, Triebold K J, Dalton D K, Stewart T A, Bloom B R. An essential role for interferon gamma in resistance to Mycobacterium tuberculosis infection. The Journal of experimental medicine 1993: 178(6): 2249-2254.
5. Algood H M, Chan J, Flynn J L. Chemokines and tuberculosis. Cytokine Growth Factor Rev 2003: 14(6): 467-477.
6. Ota M O, Mendy J F, Donkor S, Togun T, Daramy M, Gomez M P, Chegou N N, Sillah A K, Owolabi O, Kampmann B, Walzl G, Sutherland J S. Rapid diagnosis of tuberculosis using ex vivo host biomarkers in sputum. The European respiratory journal: official journal of the European Society for Clinical Respiratory Physiology 2014: 44(1): 254-257.
7. Anderson S T, Kaforou M, Brent A J, Wright V J, Banwell C M, Chagaluka G, Crampin A C, Dockrell H M, French N, Hamilton M S, Hibberd M L, Kern F, Langford P R, Ling L, Mlotha R, Ottenhoff T H, Pienaar S, Pillay V, Scott J A, Twahir H, Wilkinson R J, Coin L J, Heyderman R S, Levin M, Eley B. Diagnosis of childhood tuberculosis and host RNA expression in Africa. The New England journal of medicine 2014: 370(18): 1712-1723.

Sequence Listing

>sp|P05231|IL6_HUMAN Interleukin-6 OS = Homo sapiens GN = IL6
PE = 1 SV = 1
MNSFSTSAFGPVAFSLGLLLVLPAAFPAPVPPGEDSKDVAAPHRQPLTSSERIDKQIRYI
LDGISALRKETCNKSNMCESSKEALAENNLNLPKMAEKDGCFQSGFNEETCLVKIITG
LLEFEVYLEYLQNRFESSEEQARAVQMSTKVLIQFLQKKAKNLDAITTPDPTTNASLLT
KLQAQNQWLQDMTTHLILRSFKEFLQSSLRALRQM
>sp|P18510|IL1RA_HUMAN Interleukin-1 receptor antagonist protein
OS = Homo sapiens GN = IL1RN PE = 1 SV = 1
MEICRGLRSHLITLLLFLFHSETICRPSGRKSSKMQAFRIWDVNQKTFYLRNNQLVAG
YLQGPNVNLEEKIDVVPIEPHALFLGIHGGKMCLSCVKSGDETRLQLEAVNITDLSEN
RKQDKRFAFIRSDSGPTTSFESAACPGWFLCTAMEADQPVSLTNMPDEGVMVTKFYF
QEDE
>sp|P18510-2|IL1RA_HUMAN Isoform 2 of Interleukin-1 receptor antagonist
protein OS = Homo sapiens GN = IL1RN
MALETICRPSGRKSSKMQAFRIWDVNQKTFYLRNNQLVAGYLQGPNVNLEEKIDVVPI
EPHALFLGIHGGKMCLSCVKSGDETRLQLEAVNITDLSENRKQDKRFAFIRSDSGPTT
SFESAACPGWFLCTAMEADQPVSLTNMPDEGVMVTKFYFQEDE
>sp|P18510-3|IL1RA_HUMAN Isoform 3 of Interleukin-1 receptor antagonist
protein OS = Homo sapiens GN = IL1RN
MALADLYEEGGGGGGEGEDNADSKETICRPSGRKSSKMQAFRIWDVNQKTFYLRNN
QLVAGYLQGPNVNLEEKIDVVPIEPHALFLGIHGGKMCLSCVKSGDETRLQLEAVNIT
DLSENRKQDKRFAFIRSDSGPTTSFESAACPGWFLCTAMEADQPVSLTNMPDEGVMV
TKFYFQEDE
>sp|P18510-4|IL1RA_HUMAN Isoform 4 of Interleukin-1 receptor antagonist
protein OS = Homo sapiens GN = IL1RN
MQAFRIWDVNQKTFYLRNNQLVAGYLQGPNVNLEEKIDVVPIEPHALFLGIHGGKMC
LSCVKSGDETRLQLEAVNITDLSENRKQDKRFAFIRSDSGPTTSFESAACPGWFLCTAM
EADQPVSLTNMPDEGVMVTKFYFQEDE
>sp|P13232|IL7_HUMAN Interleukin-7 OS = Homo sapiens GN = IL7 PE = 1
SV = 1
MFHVSFRYIFGLPPLILVLLPVASSDCDIEGKDGKQYESVLMVSIDQLLDSMKEIGSNC
LNNEFNFFKRHICDANKEGMFLFRAARKLRQFLKMNSTGDFDLHLLKVSEGTTILLN
CTGQVKGRKPAALGEAQPIKSLEENKSLKEQKKLNDLCFLKRLLQEIKTCWNKILMG
TKEH
>sp|P13232-2|IL7_HUMAN Isoform 2 of Interleukin-7 OS = Homo sapiens
GN = 1L7
MFHVSFRYIFGLPPLILVLLPVASSDCDIEGKDGKQYESVLMVSIDQLLDSMKEIGSNC
LNNEFNFEKRHICDANKVKGRKPAALGEAQPIKSLEENKSLKEQKKLNDLCFLKRLL
QEIKTCWNKILMGTKEH
>sp|P13232-3|1L7_HUMAN Isoform 3 of Interleukin-7 OS = Homo sapiens
GN = 1L7
MKEIGSNCLNNEFNFFKRHICDANKEENKSLKEQKKLNDLCFLKRLLQEIKTCWNKL
MGTKEH
>sp|P10145|IL8_HUMAN Interleukin-8 OS = Homo sapiens GN = CXCL8
PE = 1 SV = 1
MTSKLAVALLAAFLISAALCEGAVLPRSAKELRCQCIKTYSKPFHPKFIKELRVIESGPH
CANTEIIVKLSDGRELCLDPKENWVQRVVEKFLKRAENS
>sp|P10145-2|IL8_HUMAN Isoform 2 of Interleukin-8 OS = Homo sapiens
GN = CXCL8
MTSKLAVALLAAFLISAALCEGAVLPRSAKELRCQCIKTYSKPFHPKFIKELRVIESGPH
CANTEIIVKLSDGRELCLDPKENWVQRVVEKAEVIDENRGMDS
>sp|P29460|IL12B_HUMAN Interleukin-12 subunit beta OS = Homo sapiens
GN = IL12B PE = 1 SV = 1
MCHQQLVISWFSLVFLASPLVAIWELKKDVYVVELDWYPDAPGEMVVLICDTPEEDG
ITWTLDQSSEVLGSGKTLTIQVKEFGDAGQYTCHKGGEVLSHSLLLLHKKEDGIWSTD
ILKDQKEPKNKTFLRCEAKNYSGRFTCWWLTTISTDLTFSVKSSRGSSDPQGVTCGAA
TLSAERVRGDNKEYEYSVECQEDSACPAAEESLPIEVMVDAVHKLKYENYLSSFFIRDII
KPDPPKNLQLKPLKNSRQVEVSWEYPDTWSTPHSYFSLTFCVQVQGKSKREKKDRVF
TDKTSATVICRKNASISVRAQDRYYSSSWSEWASVPCS
FGF-basic; P09038: FGF2
MVGVGGGDVE DVTPRPGGCQ ISGRGARGCN GIPGAAAWEA ALPRRRPRRH
PSVNPRSRAA GSPRTRGRRT EERPSGSRLG DRGRGRALPG GRLGGRGRGR
APERVGGRGR GRGTAAPRAA PAARGSRPGP AGTMAAGSIT TLPALPEDGG
SGAFPPGHFK DPKRLYCKNG GFFLRIHPDG RVDGVREKSD PHIKLQLQAE
ERGVVSIKGV CANRYLAMKE DGRLLASKCV TDECFFFERL ESNNYNTYRS
RKYTSWYVAL KRTGQYKLGS KTGPGQKAIL FLPMSAKS
>sp|P02778|CXL10_HUMAN C-X-C motif chemokine 10 OS = Homo sapiens
GN = CXCL10 PE = 1 SV = 2
MNQTAILICCLIFITLSGIQGVPLSRIVRCTCISISNQPVNPRSLEKLEIIPASQFCPRVEII
ATMKKKGEKRCLNPESKAIKNLLKAVSKERSKRSP
>sp|P15692|VEGFA_HUMAN Vascular endothelial growth factor A
OS = Homo sapiens GN = VEGFA PE = 1 SV = 2
MNFLLSWVHWSLALLLYLHHAKWSQAAPMAEGGGQNHHEVVKFMDVYQRSYCHPI
ETLVDIFQEYPDEIEYIFKPSCVPLMRCGGCCNDEGLECVPTEESNITMQIMRIKPHQG
QHIGEMSFLQHNKCECRPKKDRARQEKKSVRGKGKGQKRKRKKSRYKSWSVYVGAR
CCLMPWSLPGPHPCGPCSERRKHLFVQDPQTCKCSCKNTDSRCKARQLELNERTCRC
DKPRR
>sp|P15692-2|VEGFA_HUMAN Isoform VEGF189 of Vascular endothelial growth
factor A OS = Homo sapiens GN = VEGFA
MNFLLSWVHWSLALLLYLHHAKWSQAAPMAEGGGQNHHEVVKFMDVYQRSYCHPI
ETLVDIFQEYPDEIEYIFKPSCVPLMRCGGCCNDEGLECVPTEESNITMQIMRIKPHQG
QHIGEMSFLQHNKCECRPKKDRARQEKKSVRGKGKGQKRKRKKSRYKSWSVPCGPC
SERRKHLFVQDPQTCKCSCKNTDSRCKARQLELNERTCRCDKPRR
>sp|P15692-3|VEGFA_HUMAN Isoform VEGF183 of Vascular endothelial growth
factor A OS = Homo sapiens GN = VEGFA
MNFLLSWVHWSLALLLYLHHAKWSQAAPMAEGGGQNHHEVVKFMDVYQRSYCHPI
ETLVDIFQEYPDEIEYIFKPSCVPLMRCGGCCNDEGLECVPTEESNITMQIMRIKPHQG
QHIGEMSFLQHNKCECRPKKDRARQEKKSVRGKGKGQKRKRKKSRPCGPCSERRKH
LFVQDPQTCKCSCKNTDSRCKARQLELNERTCRCDKPRR
>sp|P15692-4|VEGFA_HUMAN Isoform VEGF165 of Vascular endothelial growth
factor A OS = Homo sapiens GN = VEGFA
MNFLLSWVHWSLALLLYLHHAKWSQAAPMAEGGGQNHHEVVKFMDVYQRSYCHPI
ETLVDIFQEYPDEIEYIFKPSCVPLMRCGGCCNDEGLECVPTEESNITMQIMRIKPHQG
QHIGEMSFLQHNKCECRPKKDRARQENPCGPCSERRKHLFVQDPQTCKCSCKNTDSR
CKARQLELNERTCRCDKPRR
>sp|P15692-5|VEGFA_HUMAN Isoform VEGF148 of Vascular endothelial growth
factor A OS = Homo sapiens GN = VEGFA
MNFLLSWVHWSLALLLYLHHAKWSQAAPMAEGGGQNHHEVVKFMDVYQRSYCHPI
ETLVDIFQEYPDEIEYIFKPSCVPLMRCGGCCNDEGLECVPTEESNITMQIMRIKPHQG
QHIGEMSFLQHNKCECRPKKDRARQENPCGPCSERRKHLFVQDPQTCKCSCKNTDSR
CKM
>sp|P15692-6|VEGFA_HUMAN Isoform VEGF145 of Vascular endothelial growth
factor A OS = Homo sapiens GN = VEGFA
MNFLLSWVHWSLALLLYLHHAKWSQAAPMAEGGGQNHHEVVKFMDVYQRSYCHPI
ETLVDIFQEYPDEIEYIFKPSCVPLMRCGGCCNDEGLECVPTEESNITMQIMRIKPHQG
QHIGEMSFLQHNKCECRPKKDRARQEKKSVRGKGKGQKRKRKKSRYKSWSVCDKPR
R
>sp|P15692-8|VEGFA_HUMAN Isoform VEGF165B of Vascular endothelial growth
factor A OS = Homo sapiens GN = VEGFA
MNFLLSWVHWSLALLLYLHHAKWSQAAPMAEGGGQNHHEVVKFMDVYQRSYCHPI
ETLVDIFQEYPDEIEYIFKPSCVPLMRCGGCCNDEGLECVPTEESNITMQIMRIKPHQG
QHIGEMSFLQHNKCECRPKKDRARQENPCGPCSERRKHLFVQDPQTCKCSCKNQDSR
CKARQLELNERTCRSLTRKD
>sp|P15692-9|VEGFA_HUMAN Isoform VEGF121 of Vascular endothelial growth
factor A OS = Homo sapiens GN = VEGFA
MNFLLSWVHWSLALLLYLHHAKWSQAAPMAEGGGQNHHEVVKFMDVYQRSYCHPI
ETLVDIFQEYPDEIEYIFKPSCVPLMRCGGCCNDEGLECVPTEESNITMQIMRIKPHQG
QHIGEMSFLQHNKCECRPKKDRARQEKCDKPRR
>sp|P15692-10|VEGFA_HUMAN Isoform VEGF111 of Vascular endothelial growth
factor A OS = Homo sapiens GN = VEGFA
MNFLLSWVHWSLALLLYLHHAKWSQAAPMAEGGGQNHHEVVKFMDVYQRSYCHPI
ETLVDIFQEYPDEIEYIFKPSCVPLMRCGGCCNDEGLECVPTEESNITMQIMRIKPHQG
QHIGEMSFLQHNKCECRCDKPRR
>sp|P15692-11|VEGFA_HUMAN Isoform L-VEGF165 of Vascular endothelial
growth factor A OS = Homo sapiens GN = VEGFA
MTDRQTDTAPSPSYHLLPGRRRTVDAAASRGQGPEPAPGGGVEGVGARGVALKLFVQ
LLGCSREGGAVVRAGEAEPSGAARSASSGREEPQPEEGEEEEEKEEERGPQWRLGAR
KPGSWTGEAAVCADSAPAARAPQALARASGRGGRVARRGAEESGPPHSPSRRGSASR
AGPGRASETMNFLLSWVHWSLALLLYLHHAKWSQAAPMAEGGGQNHHEVVKFMD
VYQRSYCHPIETLVDIFQEYPDEIEYIFKPSCVPLMRCGGCCNDEGLECVPTEESNITM
QIMRIKPHQGQHIGEMSFLQHNKCECRPKKDRARQENPCGPCSERRKHLFVQDPQT
CKCSCKNTDSRCKARQLELNERTCRCDKPRR
>sp|P15692-12|VEGFA_HUMAN Isoform L-VEGF121 of Vascular endothelial
growth factor A OS = Homo sapiens GN = VEGFA
MTDRQTDTAPSPSYHLLPGRRRTVDAAASRGQGPEPAPGGGVEGVGARGVALKLFVQ
LLGCSRFGGAVVRAGEAEPSGAARSASSGREEPQPEEGEEEEEKEEERGPQWRLGAR
KPGSWTGEAAVCADSAPAARAPQALARASGRGGRVARRGAEESGPPHSPSRRGSASR
AGPGRASETMNFLLSWVHWSLALLLYLHHAKWSQAAPMAEGGGQNHHEVVKFMD
VYQRSYCHPIETLVDIFQEYPDEIEYIFKPSCVPLMRCGGCCNDEGLECVPTEESNITM
QIMRIKPHQGQHIGEMSFLQHNKCECRPKKDRARQEKCDKPRR
>sp|P15692-13|VEGFA_HUMAN Isoform L-VEGF189 of Vascular endothelial
growth factor A OS = Homo sapiens GN = VEGFA
MTDRQTDTAPSPSYHLLPGRRRTVDAAASRGQGPEPAPGGGVEGVGARGVALKLFVQ
LLGCSRFGGAVVRAGEAEPSGAARSASSGREEPQPEEGEEEEEKEEERGPQWRLGAR
KPGSWTGEAAVCADSAPAARAPQALARASGRGGRVARRGAEESGPPHSPSRRGSASR
AGPGRASETMNFLLSWVHWSLALLLYLHHAKWSQAAPMAEGGGQNHHEVVKFMD
VYQRSYCHPIETLVDIFQEYPDEIEYIFKPSCVPLMRCGGCCNDEGLECVPTEESNITM
QIMRIKPHQGQHIGEMSFLQHNKCECRPKKDRARQEKKSVRGKGKGQKRKRKKSRY
KSWSVPCGPCSERRKHLFVQDPQICKCSCKNTDSRCKARQLELNERTCRCDKPRR
>sp|P15692-14|VEGFA_HUMAN Isoform L-VEGF206 of Vascular endothelial
growth factor A OS = Homo sapiens GN = VEGFA
MTDRQTDTAPSPSYHLLPGRRRTVDAAASRGQGPEPAPGGGVEGVGARGVALKLFVQ
LLGCSRFGGAVVRAGEAEPSGAARSASSGREEPQPEEGEEEEEKEEERGPQWRLGAR
KPGSWTGEAAVCADSAPAARAPQALARASGRGGRVARRGAEESGPPHSPSRRGSASR
AGPGRASETMNFLLSWVHWSLALLLYLHHAKWSQAAPMAEGGGQNHHEVVKFMD
VYQRSYCHPIETLVDIFQEYPDEIEYIFKPSCVPLMRCGGCCNDEGLECVPTEESNITM
QIMRIKPHQGQHIGEMSFLQHNKCECRPKKDRARQEKKSVRGKGKGQKRKRKKSRY
KSWSVYVGARCCLMPWSLPGPHPCGPCSERRKHLFVQDPQICKCSCKNIDSRCKARQ
LELNERTCRCDKPRR
>sp|P15692-15|VEGFA_HUMAN Isoform 15 of Vascular endothelial
growth factor A OS = Homo sapiens GN = VEGFA
MTDRQTDTAPSPSYHLLPGRRRTVDAAASRGQGPEPAPGGGVEGVGARGVALKLFVQ
LLGCSRFGGAVVRAGEAEPSGAARSASSGREEPQPEEGEEEEEKEEERGPQWRLGAR
KPGSWTGEAAVCADSAPAARAPQALARASGRGGRVARRGAEESGPPHSPSRRGSASR
AGPGRASETMNFLLSWVHWSLALLLYLHHAKWSQAAPMAEGGGQNHHEVVKFMD
VYQRSYCHPIETLVDIFQEYPDEIEYIFKPSCVPLMRCGGCCNDEGLECVPTEESNITM
QIMRIKPHQGQHIGEMSFLQHNKCECRPKKDRARQENPCGPCSERRKHLFVQDPQT
CKCSCKNTDSRCKARQLELNERTCRSLTRKD
>sp|P15692-16|VEGFA_HUMAN Isoform 16 of Vascular endothelial growth
factor A OS = Homo sapiens GN = VEGFA
MTDRQTDTAPSPSYHLLPGRRRTVDAAASRGQGPEPAPGGGVEGVGARGVALKLFVQ
LLGCSRFGGAVVRAGEAEPSGAARSASSGREEPQPEEGEEEEEKEEERGPQWRLGAR
KPGSWTGEAAVCADSAPAARAPQALARASGRGGRVARRGAEESGPPHSPSRRGSASR
AGPGRASETMNFLLSWVHWSLALLLYLHHAKWSQAAPMAEGGGQNHHEVVKFMD
VYQRSYCHPIETLVDIFQEYPDEIEYIFKPSCVPLMRCGGCCNDEGLECVPTEESNITM
QIMRIKPHQGQHIGEMSFLQHNKCECRPKKDRARQEKKSVRGKGKGQKRKRKKSRP
CGPCSERRKHLFVQDPQTCKCSCKNTDSRCKARQLELNERTCRCDKPRR
>sp|P15692-17|VEGFA_HUMAN Isoform 17 of Vascular endothelial growth
factor A OS = Homo sapiens GN = VEGFA
MTDRQTDTAPSPSYHLLPGRRRTVDAAASRGQGPEPAPGGGVEGVGARGVALKLFVQ
LLGCSRFGGAVVRAGEAEPSGAARSASSGREEMPEEGEEEEEKEEERGPQWRLGAR
KPGSWTGEAAVCADSAPAARAPQALARASGRGGRVARRGAEESGPPHSPSRRGSASR
AGPGRASETMNFLLSWVHWSLALLLYLHHAKWSQAAPMAEGGGQNHHEVVKFMD
VYQRSYCHPIETLVDIFQEYPDEIEYIFKPSCVPLMRCGGCCNDEGLECVPTEESNITM
QIMRIKPHQGQHIGEMSFLQHNKCECRPKKDRARQENPCGPCSERRKHLFVQDPQT
CKCSCKNIDSRCKM
>sp|P15692-18|VEGFA_HUMAN Isoform 18 of Vascular endothelial growth
factor A OS = Homo sapiens GN = VEGFA
MTDRQTDTAPSPSYHLLPGRRRTVDAAASRGQGPEPAPGGGVEGVGARGVALKLFVQ
LLGCSRFGGAVVRAGEAEPSGAARSASSGREEPQPEEGEEEEEKEEERGPQWRLGAR
KPGSWTGEAAVCADSAPAARAPQALARASGRGGRVARRGAEESGPPHSPSRRGSASR
AGPGRASETMNFLLSWVHWSLALLLYLHHAKWSQAAPMAEGGGQNHHEVVKFMD
VYQRSYCHPIETLVDIFQEYPDEIEYIFKPSCVPLMRCGGCCNDEGLECVPTEESNITM
QIMRIKPHQGQHIGEMSFLQHNKCECRCDKPRR

What is claimed:

1-26. (canceled)

27. A method of treating tuberculosis (TB) in a subject, the method comprising:
a. selecting a patient for treatment based on the presence or absence of each of IL-1ra, IL-6, IL-7, IL-8, IL-12p70, FGF-basic, IP-10, and VEGF in a sample from said subject; and
b. administering a treatment regimen for TB to said subject only when the presence of each of IL-1ra, IL-6, IL-7, IL-8, IL-12p70, FGF-basic, IP-10, and VEGF is determined.

28. The method of claim 27, wherein step (a) comprises the steps of:
a. providing a sample from said subject, said sample being selected from the group consisting of: blood, serum and plasma;
b. determining the concentration in said sample of IL-1ra, IL-6, IL-7, IL-8, IL-12p70, FGF-basic, IP-10, and VEGF;
c. converting each concentration determined in (b) into a decile value; and
d. converting each decile value into a binary presence or absence by comparing the decile values of (c) to the following specific quantile cut-off values:

| | Specific quantile cut- |
|---|---|
| | off value |
| IL-1ra | 3 |
| IL-6 | 6 |
| IL-7 | 8 |
| IL-8 | 9 |
| IL-12p70 | 9 |
| FGF-basic | 3 |
| IP-10 | 4 |
| VEGF | 9 |

wherein a decile value matching or exceeding the specific quantile cut-off value is converted into the binary presence of IL-1ra, IL-6, IL-7, IL-8, IL-12p70, FGF-basic, IP-10, and VEGF, and a decile value lower than the specific quantile cut-off value is converted into the binary absence of IL-1ra, IL-6, IL-7, IL-8, IL-12p70, FGF-basic, IP-10, and VEGF.

29. The method according to claim 28, wherein step (c) converting each concentration determined in (b) into a decile value comprises the steps of:
a. comparing the concentration of IL-1ra, IL-6, IL-7, IL-8, IL-12p70, FGF-basic, IP-10, and VEGF determined in (b) to a reference frequency distribution of concentrations of s IL-1ra, IL-6, IL-7, IL-8, IL-12p70, FGF-basic, IP-10, and VEGF; and
b. reading out the decile value from the frequency distribution for the concentration of said IL-1ra, IL-6, IL-7, IL-8, IL-12p70, FGF-basic, IP-10, and VEGF.

30. The method according to claim 27, wherein said sample is a sample of serum or plasma.

31. The method according to claim 30, wherein said serum or plasma is essentially cell free.

32. The method according to claim 27, wherein the subject is 16 years old or younger.

33. The method according to claim 27, wherein the subject is 2 years old or older.

34. The method according to claim 27, wherein the subject is 5 to 15 years old.

35. The method according to claim 28, wherein said method further comprises selecting a patient for treatment based on the presence or absence of EC-stimulated VEGF and the specific quantile cut-off value for EC-stimulated VEGF is 2.

36. The method of claim 27, wherein the treatment regimen is:
a. two months of treatment with Isoniazid, Rifampicin, Pyrazinamide and Ethambutol followed by four months of treatment with Isoniazid and Rifampicin;
b. two months of treatment with Isoniazid, Rifampicin, Pyrazinamide and Ethambutol followed by four months of treatment with Isoniazid, Rifampicin and Ethambutol; or
c. six months of treatment with Rifampicin.

37. The method of claim 27, wherein the treatment regimen is two months of treatment with Isoniazid, Rifampicin, Pyrazinamide and Ethambutol followed by four months of treatment with Isoniazid and Rifampicin, and the subject is dosed at least three times per week or daily during the two months of treatment with Isoniazid, Rifampicin, Pyrazinamide and Ethambutol.

38. A method for selecting a treatment regimen, said process comprising the steps of:
a. providing a sample from a subject, said sample being selected from the group consisting of: blood, serum and plasma;
b. determining the concentration in said sample of each of IL-1ra, IL-6, IL-7, IL-8, IL-12p70, FGF-basic, IP-10, and VEGF;
c. converting each concentration determined in (b) into a decile value;
d. converting each decile value into a binary presence or absence by comparing the decile values of (c) to the following specific quantile cut-off values wherein a decile value matching or exceeding the specific quantile cut-off value is converted into the binary presence of IL-1ra, IL-6, IL-7, IL-8, IL-12p70, FGF-basic, IP-10, and VEGF, and a decile value lower than the specific quantile cut-off value is converted into the binary absence of t IL-1ra, IL-6, IL-7, IL-8, IL-12p70, FGF-basic, IP-10, and VEGF:

| | Specific quantile cut- |
|---|---|
| | off value |
| IL-1ra | 3 |
| IL-6 | 6 |
| IL-7 | 8 |
| IL-8 | 9 |
| IL-12p70 | 9 |
| FGF-basic | 3 |
| IP-10 | 4 |
| VEGF | 9 |

e. selecting a tuberculosis treatment regimen based on the presence of each of IL-1ra, IL-6, IL-7, IL-8, IL-12p70, FGF-basic, IP-10, and VEGF in the sample; and from said subject; and
f. optionally administering the tuberculosis treatment regimen to said subject.

39. The method of claim 38, wherein the treatment regimen is:
a. two months of treatment with Isoniazid, Rifampicin, Pyrazinamide and Ethambutol followed by four months of treatment with Isoniazid and Rifampicin;
b. two months of treatment with Isoniazid, Rifampicin, Pyrazinamide and Ethambutol followed by four months of treatment with Isoniazid, Rifampicin and Ethambutol; or
c. six months of treatment with Rifampicin.

40. The method of claim 38, wherein the treatment regimen is two months of treatment with Isoniazid, Rifampicin, Pyrazinamide and Ethambutol followed by four months of treatment with Isoniazid and Rifampicin, and the subject is dosed at least three times per week or daily during the two months of treatment with Isoniazid, Rifampicin, Pyrazinamide and Ethambutol.

41. A kit comprising a reagent(s) for the specific detection of each of IL-ra, IL-6, IL-7, IL-8, IL-12p70, FGF-basic, IP-10, VEGF, and optionally, EC-stimulated VEGF.

42. A device comprising an array of materials which together are capable of specifically detecting each of: IL-1ra, IL-6, IL-7, IL-8, IL-12p70, FGF-basic, IP-10, and VEGF and optionally EC-stimulated VEGF, each material within the array being capable of specifically detecting one of said IL-1ra, IL-6, IL-7, IL-8, IL-12p70, FGF-basic, IP-10, and VEGF and optionally EC-stimulated VEGF.

43. The device of claim 42, wherein the detection is specifically detecting a protein or specifically detecting an mRNA.

44. The device of claim 42 which is a lateral flow device.

45. A computer program product operable, when executed on a computer, to perform the method steps of claim 27.

46. An apparatus comprising logic configured to carry out the method of claim 27.

47. A method for the diagnosis of tuberculosis (TB) in a subject, the method comprising:
a. providing a sample from said subject, said sample being selected from the group consisting of: blood, serum and plasma;
b. determining the concentration in said sample of IL-1ra, IL-6, IL-7, IL-8, IL-12p70, FGF-basic, IP-10, and VEGF;
c. converting each concentration determined in (b) into a decile value; and
d. converting each decile value into a binary presence or absence by comparing the decile values of (c) to the following specific quantile cut-off values:

| | Specific quantile cut- |
|---|---|
| | off value |
| IL-1ra | 3 |
| IL-6 | 6 |
| IL-7 | 8 |
| IL-8 | 9 |
| IL-12p70 | 9 |
| FGF-basic | 3 |
| IP-10 | 4 |
| VEGF | 9 |

wherein a decile value matching or exceeding the specific quantile cut-off value is converted into the binary presence of IL-1ra, IL-6, IL-7, IL-8, IL-12p70, FGF-basic, IP-10, and VEGF, and a decile value lower than the specific quantile cut-off value is converted into the binary absence of IL-1ra, IL-6, IL-7, IL-8, IL-12p70, FGF-basic, IP-10, and VEGF, wherein detecting the presence of each of IL-1ra, IL-6, IL-7, IL-8, IL-12p70, FGF-basic, IP-10, and VEGF indicates said subject has TB.
